(19) United States
(12) Patent Application Publication
(10) Pub. No.: US 2025/0205268 A1
(43) Pub. Date: Jun. 26, 2025

(54) TREATMENT OF VIRUSES WITH ANTIVIRAL NUCLEOSIDES

(71) Applicants: MERCK SHARP & DOHME LLC, Rahway, NJ (US); EMORY UNIVERSITY, Atlanta, GA (US)

(72) Inventors: Carisa Stadlman DE ANDA, Rahway, NJ (US); Wayne G. HOLMAN, Miami, FL (US); Yoshihiko MURATA, Rahway, NJ (US); George PAINTER, Atlanta, GA (US); Gwendolyn PAINTER, Oviedo, FL (US); Michelle L. BROWN, Rahway, NJ (US); Matthew G. JOHNSON, Rahway, NJ (US); Myra Christine Wooley POPEJOY, Rahway, NJ (US); Julie STONE, Rahway, NJ (US)

(73) Assignees: MERCK SHARP & DOHME LLC, Rahway, NJ (US); EMORY UNIVERSITY, Atlanta, GA (US)

(21) Appl. No.: 18/702,922

(22) PCT Filed: Oct. 20, 2022
(86) PCT No.: PCT/US2022/078467

§ 371 Date: Apr. 19, 2024

Related U.S. Application Data
(60) Provisional application No. 63/257,649, filed on Oct. 20, 2021.

(51) Int. Cl. A61K 31/7072; A61K 9/00; A61K 9/48; A61P 31/14

(52) U.S. Cl. CPC A61K 31/7072; A61K 9/0053; A61K 9/48; A61P 31/14

(57) ABSTRACT

Therapies comprising administering at least one antiviral nucleoside, and the use of such therapies in the treatment of viral infections, such as infection by Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, or Zika virus, are disclosed herein.

This application claims priority from U.S. Patent Application No. 63/257,649 filed Oct. 20, 2021, and U.S. Patent Application No. 63/275,647 filed Nov. 4, 2021, which are hereby incorporated by reference in their entirety.

The present disclosure relates to uses of therapeutic compounds to treat viruses, including human coronaviruses such as SARS-CoV-2. In particular, this disclosure relates to therapies comprising at least one antiviral nucleoside that is useful as an antiviral.

Viral infections, such as infections caused by Eastern Equine Encephalitis Virus (EEEV), Western Equine Encephalitis Virus (WEEV), and Venezuelan Equine Encephalitis Virus (VEEV), Chikungunya fever virus (CHIK), Ebola virus, influenza virus, respiratory syncytial virus (RSV), Zika virus, and coronaviruses, such as Severe Acute Respiratory Syndrome Coronavirus (SARS-COV), Middle East Respiratory Syndrome Coronavirus (MERS-CoV), and, most recently, SARS-CoV-2 (also known as 2019-nCoV), continue to cause illnesses, which can be mild to severe to life-threatening and fatal, across the globe.

EEEV, WEEV, VEEV, and CHIK virus are vector-borne viruses (family Togaviridae, genus Alphavirus) that can be transmitted to humans through mosquito bites. The equine encephalitis viruses are CDC Category B pathogens, and the CHIK virus is Category C.

Coronaviruses cause a large percentage of respiratory illness in humans, which can be severe or life-threatening. SARS-CoV-1, which emerged in 2002, has caused at least 8439 human illnesses globally and at least 812 deaths (WHO Cumulative Number of Reported Probable Cases of SARS, From 1 Nov. 2002 To 4 Jul. 2003, downloaded from https://www.who.int/csr/sars/country/2003_07_04/en/, on Aug. 12, 2020). Similarly, MERS-CoV emerged in 2012 and has caused at least 2519 human illnesses globally and at least 866 deaths (WHO Middle East respiratory syndrome, MERS situation update, January 2020, downloaded from http://www.emro.who.int/health-topics/mers-cov/mers-outbreaks.html, on Aug. 12, 2020). More recently, SARS-CoV-2 emerged in 2019, and it has caused at least 237,655,302 human illnesses globally and at least 4,846,981 deaths (COVID-19 Weekly Operational Update, Issue No. 75, published 12 Oct. 2021, downloaded from https://www.who.int/publications/m/item/weekly-operational-update-on-covid-19—12-october-2021, on Oct. 19, 2021). SARS-CoV-2 causes the disease referred to as COVID-19, which can include severe respiratory disease and systemic disease manifestations in humans. SARS-CoV-2 infection is also associated with mental and neurological symptoms that can include delirium or encephalopathy, agitation, stroke, meningoencephalitis, impaired sense of smell or taste, anxiety, depression, and sleep problems, and these neurological symptoms can occur in the absence of respiratory symptoms (see Clinical management of COVID-19 (Interim guidance, 27 May 2020), downloaded from https://www.who.int/publications/i/item/clinical-management-of-covid-19, on Sep. 15, 2020). Additional studies are needed to further characterize the SARS-CoV-2 virus and to identify ways to prevent and treat the COVID-19 disease, as well as diseases caused by other human coronaviruses.

Many patients with COVID-19 recover with no or minimal medical intervention. However, clinical progression to severe disease severely impacts both patients and healthcare systems, increasing an individual's risks of requiring mechanical ventilation and of death and potentially overburdening hospital capacity and available healthcare resources during COVID-19 surges. Reducing the number of patients requiring hospitalization for COVID-19 is therefore critical. Vaccination remains by far the most important medical intervention available to reduce the risk of hospitalization or death from COVID-19. However, early treatment soon after symptom onset has also been shown as effective. The monoclonal antibodies bamlanivimab/etesevimab, casirivimab/imdevimab, and sotrovimab are currently the only treatments authorized for at-risk outpatients with COVID-19. Because monoclonal antibodies require administration via infusion or injection in a medical setting, a direct-acting, oral agent that can be self-administered at home after diagnosis may be more practical for non-hospitalized patients and would be an important new tool in treating COVID-19 caused by SARS-CoV-2.

β-D-N(4)-hydroxycytidine (NHC, 1-((2R,3R,4S,5R)-3,4-dihydroxy-5-(hydroxymethyl)tetrahydrofuran-2-yl)-4-(hydroxyamino)pyrimidin-2 (1H)-one) was found to have antipestivirus and antihepacivirus activities. ANTIMICROB AGENTS CHEMOTHER, 2003, 47 (1): 244-54. β-D-N(4)-hydroxycytidine,

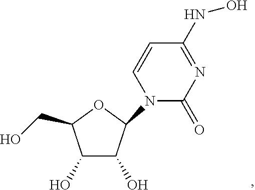 [SMILES: O=c1nc(NO)ccn1[C@@H]1O[C@H](CO)[C@@H](O)[C@H]1O]

derivatives thereof, and methods for making and using the same are illustrated in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety.

In view of the potential of viral infections to cause illness and death, there remains a need for new therapies and dosing regimens that can be used to treat viral infections, and for therapies that effectively treat viral infections.

Embodiments of the disclosure include therapies comprising at least one antiviral nucleoside. In particular, embodiments of the disclosure include therapies comprising Compound A, which may also be disclosed as molnupiravir, as MK-4482 or EIDD-2801, as CAS Number 2349386-89-4, by its tautomers N-hydroxycytidine 5′-(2-methylpropanoate) and uridine 4-oxime 5′-(2-methylpropanoate), by IUPAC names {(2R,3S,4R,5R)-3,4-dihydroxy-5-[4-(hydroxyimino)-2-oxo-3,4-dihydropyrimidin-1 (2H)-yl]oxolan-2-yl}methyl 2-methylpropanoate) and {(2R,3S,4R,5R)-3,4-dihydroxy-5-[4-(hydroxyamino)-2-oxopyrimidin-1-yl]oxolan-2-yl}methyl 2-methylpropanoate), and/or by structure, such as:

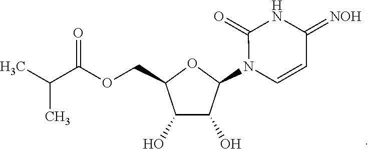

Pharmaceutically acceptable salts, derivatives, tautomers, isomers, and prodrugs of Compound A are included within the scope of the present disclosure. Molnupiravir has been disclosed in PCT International Patent Application No. PCT/US2018/064503, which published as PCT International Patent Application Publication No. WO2019/113462, and PCT International Patent Application No. PCT/US2021/016984, which published as PCT International Patent Application Publication No. WO2021/159044, as well as particular forms as disclosed in PCT International Patent Application No. PCT/US2021/048054, filed Aug. 27, 2021.

Another embodiment includes a method of treating a viral infection in a subject in need thereof, comprising administering a therapy comprising at least one antiviral nucleoside.

Other embodiments, aspects and features of the present disclosure are either further described in or will be apparent from the ensuing description, examples, and claims.

### DESCRIPTION OF FIGURES

FIG. 1 shows Time-to-Event Analysis of Hospitalization or Death through Day 29 in the Modified Intention-to-Treat (MITT) Population as Kaplan-Meier curves with 95% confidence intervals (I bars). X indicates censored values.

FIG. 2 shows Incidence of Hospitalization or Death at Day 29 in the MITT Population for the primary end point in key subgroups of the modified intention-to-treat population.

FIG. 3 shows mean change in high-sensitivity C-reactive protein (CRP) concentration through day 29 (safety population). Error bars represent 95% CIs. EOT=end of therapy.

FIG. 4 shows Mean Change in Oxygen Saturation (SpO₂) through day 29 (safety population). Error bars represent 95% CIs. EOT=end of therapy.

FIG. 5 shows Respiratory Interventions through day 29 in the MITT population (top) and the hospitalized MITT population (bottom). Respiratory interventions included conventional oxygen (O₂), high-flow (HF) heated and humidified device, noninvasive mechanical ventilation (MV), and invasive MV. Error bars represent 95% CIs.

FIG. 6 shows acute care visits and COVID-19-related acute care visits through day 29 in the MITT population.

### DETAILED DESCRIPTION

### Abbreviations

ACTT-1 Adaptive COVID-19 Treatment Trial
AE Adverse event
AUC Area under the curve
BID Twice daily
C8hr Plasma concentration value collected at 8 hours
Cₘₐₓ Maximum plasma concentration
Ctrl Control
C_{trough} Lowest plasma concentration
DILI Drug-induced liver injury
DLT Dose limiting toxicity
DMAP 4-dimethylaminopyridine
ECG Electrocardiogram
ECI Event of clinical interest
eDMC External data monitoring committee
equiv Equivalents
H1N1 Hemagglutinin Type 1 and Neuraminidase Type 1 (influenza strain)
IA Interim analysis
kg Kilogram
L Liter
LFT Liver function test
LFU Late follow-up visit
mg Milligram
MITT Modified intent-to-treat (population)
mL Milliliter
mm Millimeter
mm³ Cubic millimeter, 0.001 mL
mol mole
MPK Milligram per kilogram
MTBE Methyl tert-butyl ether
MTD Maximum tolerated dose
n Number of subjects in a treatment group
NHC N-hydroxycytidine
PBMC Peripheral blood mononuclear cells
PCR Polymerase chain reaction
PK Pharmacokinetic
PR Partial response
p-values Calculated probability
Q12H Administered once every 12 hours
QD Once daily
SAE Severe Adverse Event
SEM Standard error of the mean
siDMC Standing internal data monitoring committee
t_{1/2} Half life
tₘₐₓ Time to maximum plasma concentration
ULN Upper limit of normal
VL Viral load
WHO World Health Organization

Additional abbreviations may be defined throughout this disclosure.

### Definitions

Certain technical and scientific terms are specifically defined below. Unless specifically defined elsewhere in this document, all other technical and scientific terms used herein have the meaning commonly understood by one of ordinary skill in the art to which this disclosure relates.

As used herein, including the appended claims, the singular forms of words such as “a,” “an,” and “the,” include their corresponding plural references unless the context clearly dictates otherwise.

The terms “administration of” and “administering” a compound should be understood to include providing a compound described herein, or a pharmaceutically acceptable salt thereof, and compositions of the foregoing to a subject.

As used herein, the terms “at least one” item and “one or more” item each include a single item selected from the list as well as mixtures of two or more items selected from the list.

The term “pharmaceutically acceptable carrier” refers to any inactive substance that is suitable for use in a formulation for the delivery of a therapeutic agent. A carrier may be an antiadherent, binder, coating, disintegrant, filler or diluent, lubricant, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

The term “subject” (alternatively “patient” or “participant”, as in a clinical trial participant) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), Lagomorpha (e.g., rabbits), rodents (e.g., rats or mice), Procyon lotor (e.g., raccoons). In particular embodiments, the subject is human.

The term “subject in need thereof” as used herein refers to a subject diagnosed with, or suspected of having, a viral infection, such as infection by SARS-CoV-2 (either symptomatic or asymptomatic); a subject at risk of being exposed to a viral infection, such as at risk of being exposed to a viral infection, such as infection by SARS-CoV-2 (such as, for example, health care workers who may be at risk of exposure to SARS-CoV-2); a subject exposed to a viral infection, such as infection by SARS-CoV-2 (such as household contacts of COVID-19 patients or asymptomatic patients infected with SARS-CoV-2), as defined herein.

As used herein, the term “COVID-19” refers to the disease caused by SARS-CoV-2 infection. Subjects infected with SARS-CoV-2 who have developed symptoms are considered to have COVID-19.

Certain subjects may be considered to be at increased risk for severe illness from COVID-19. Such individuals may have one or more underlying medical conditions associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding individuals on dialysis or who have reduced estimated glomerular filtration rate (eGFR)<30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

As used herein, the terms “prophylaxis” and “antiviral prophylaxis” refer to all processes intended to prevent disease. Prophylaxis may occur prior to exposure to a viral infection, such as infection by SARS-CoV-2 (pre-exposure, for example, in health care workers who may be exposed to such infection) or after a potential exposure to a viral infection, such as infection by SARS-CoV-2 (post-exposure, for example, in household members or caregivers of symptomatic or asymptomatic patients infected with SARS-CoV-2).

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, pulmonary (inhalation), or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art.

The term “simultaneous administration” as used herein in relation to the administration of medicaments refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

Unless expressly stated to the contrary, all ranges cited herein are inclusive; i.e., the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, and ±10% and their numerical equivalents. “About” when used to modify a numerically defined parameter (e.g., the dose of an antiviral nucleoside, or the length of treatment time with a combination therapy described herein) means that the parameter may vary by as much as 10% below or above the stated numerical value for that parameter; where appropriate, the stated parameter may be rounded to the nearest whole number. For example, a dose of about 5 mg/kg may vary between 4.5 mg/kg and 5.5 mg/kg. In addition, the term “or,” as used herein, denotes alternatives that may, where appropriate, be combined; that is, the term “or” includes each listed alternative separately as well as their combination.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

It is understood that wherever embodiments are described herein with the language “comprising,” otherwise analogous embodiments described in terms of “consisting of” and/or “consisting essentially of” are also provided.

Exemplary methods and materials are described herein, although methods and materials similar or equivalent to those described herein can also be used in the practice or testing of the present disclosure. The materials, methods, and examples are illustrative only and not intended to be limiting.

The present disclosure relates to methods of treating a viral infection as defined herein, wherein the method comprises administering to a subject in need thereof a therapy that comprises an antiviral nucleoside.

The present disclosure relates to methods of treating a viral infection, wherein the method comprises administering to a subject in need thereof a therapy that comprises an antiviral nucleoside; wherein the viral infection is an infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In certain specific embodiments, the virus is SARS-CoV-2.

Still further, the present disclosure relates to methods of treating a viral infection, wherein the method comprises administering to a subject in need thereof a therapeutically effective amount of an antiviral nucleoside; wherein the viral infection is an infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridac virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In certain specific embodiments, the virus is SARS-CoV-2.

The present disclosure relates to methods of providing antiviral prophylaxis, wherein the method comprises administering to a subject in need thereof a therapy that comprises an antiviral nucleoside; wherein the virus is selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

The present disclosure additionally relates to methods of providing antiviral prophylaxis, wherein the method comprises administering to a subject in need thereof a therapeutically effective amount of an antiviral nucleoside; wherein the virus is selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In certain specific embodiments, the virus is SARS-CoV-2.

In COVID-19 caused by SARS-CoV-2 viral infection, SARS-CoV-2 viral loads are understood to be highest early in the course of disease, present 1-2 days prior to symptom onset, and persist for 7-12 days in moderate cases and up to 2 weeks in severe cases. As the host inflammatory response predominates during later stages of disease as COVID-19 progresses, treatment with antiviral therapy is likely to have a greater benefit with early treatment rather than delayed treatment >5 days after signs/symptoms onset, especially in a non-hospitalized setting, where patients are often earlier in the course of their disease, and intervention can occur sooner than in the hospital setting.

### Antiviral Nucleosides

As used herein, the term “antiviral nucleosides” means any nucleoside chemical compound that exhibits antiviral activity, and in particular, the antiviral nucleosides as disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, and PCT International Patent Application No. PCT/US2021/016984, which published as PCT International Patent Application Publication No. WO2021/159044, which are incorporated herein by reference in their entirety. In particular, the term “antiviral nucleosides” includes Compound A, pharmaceutically acceptable salts, derivatives, tautomers, isomers, and prodrugs of Compound A, and mixtures of any of the foregoing, as well as particular forms as disclosed in PCT International Patent Application No. PCT/US2021/048054, filed Aug. 27, 2021:

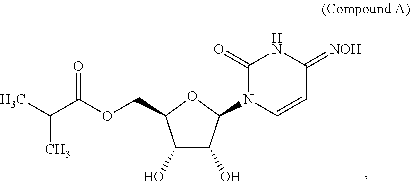

and forms of Compound A, including tautomers thereof, and including as disclosed in PCT International Patent Application No. PCT/US2018/064503, which published as PCT International Patent Application Publication No. WO2019/113462, and PCT International Patent Application No. PCT/US2021/016984, which published as PCT International Patent Application Publication No. WO2021/159044. Those skilled in the art will recognize that certain compounds, such as Compound A and other compounds containing certain heteroatoms and double or triple bonds, can be tautomers, structural isomers that readily interconvert. Thus, tautomeric compounds can be drawn in a number of different ways that are equivalent. A non-limiting example of such tautomers include those exemplified below.

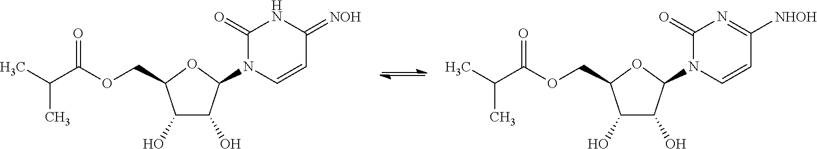

For clarity, the term “antiviral nucleosides” includes pharmaceutically acceptable salts, derivatives, tautomers, isomers, and prodrugs of such compounds, and mixtures thereof. Antiviral nucleosides, and particularly Compound A, may be used in the therapeutic combinations of this disclosure.

In embodiments, the antiviral nucleoside is Compound A.

An additional aspect of this embodiment relates to a pharmaceutical composition, said pharmaceutical composition comprising (a) Compound A or a pharmaceutically acceptable salt thereof; and (b) a pharmaceutically acceptable carrier.

An additional aspect of this embodiment relates to methods of inducing an antiviral response in a subject, comprising administering a therapeutically effective amount of Compound A or a pharmaceutically acceptable salt thereof to the subject.

An additional aspect of this embodiment relates to methods of inducing an antiviral response in a subject, comprising administering a therapeutically effective amount of a composition described above to the subject.

Additional embodiments of this disclosure relate to uses of Compound A and pharmaceutically acceptable salts thereof. Compound A may be useful as an agent to induce antiviral responses, and/or to treat a viral infection.

In embodiments, the antiviral nucleoside is a mixture of Compound A and tautomers thereof.

In embodiments, the antiviral nucleoside is a mixture of Compound A, pharmaceutically acceptable salts of Compound A, tautomers of Compound A, and pharmaceutically acceptable salts of tautomers of Compound A.

An additional aspect of this embodiment relates to a pharmaceutical composition, said pharmaceutical composition comprising (a) a mixture of Compound A and tautomers thereof, or pharmaceutically acceptable salts thereof; and (b) a pharmaceutically acceptable carrier.

An additional aspect of this embodiment relates to methods of inducing an antiviral response in a subject, comprising administering a therapeutically effective amount of a mixture of Compound A and tautomers thereof, or pharmaceutically acceptable salts thereof, to the subject.

An additional aspect of this embodiment relates to methods of inducing an antiviral response in a subject, comprising administering a therapeutically effective amount of a composition described above to the subject.

### Salts

The compounds of the present disclosure can be employed in the form of pharmaceutically acceptable salts. Those skilled in the art will recognize those instances in which the compounds of the disclosure may form salts. Examples of such compounds are described herein by reference to possible salts. Such reference is for illustration only. Pharmaceutically acceptable salts can be used with compounds for treating patients. Non-pharmaceutical salts may, however, be useful in the preparation of intermediate compounds.

The term “pharmaceutically acceptable salt” refers to a salt (including an inner salt such as a zwitterion) that possesses effectiveness similar to the parent compound and that is not biologically or otherwise undesirable (e.g., is neither toxic nor otherwise deleterious to the recipient thereof). Thus, an embodiment of the disclosure provides pharmaceutically acceptable salts of the compounds of the disclosure. The term “salt(s)”, as employed herein, denotes any of the following: acidic salts formed with inorganic and/or organic acids, as well as basic salts formed with inorganic and/or organic bases. Salts of compounds of the disclosure may be formed by methods known to those of ordinary skill in the art, for example, by reacting a compound of the disclosure with an amount of acid or base, such as an equivalent amount, in a medium such as one in which the salt precipitates or in aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al., Camille G. (eds.), Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the disclosure and all acid and base salts are considered equivalent to the free forms of the corresponding compounds for purposes of the disclosure.

In addition, when a compound contains both a basic moiety, such as, but not limited to an aliphatic primary, secondary, tertiary, or cyclic amine, an aromatic or heteroaryl amine, pyridine or imidazole, and an acidic moiety, such as, but not limited to tetrazole or carboxylic acid, zwitterions (“inner salts”) may be formed and are included within the terms “salt(s)” as used herein. It is understood that certain antiviral nucleosides may exist in zwitterionic form, having both anionic and cationic centers within the same compound and a net neutral charge. Such zwitterions are included within the disclosure.

### Methods of Preparing Compounds

Methods for preparing antiviral nucleosides, such as Compound A, and pharmaceutically acceptable salts thereof, are described herein. Starting materials and intermediates are purchased from commercial sources, made from known procedures, or are otherwise illustrated. In some cases, the order of carrying out the steps of the reaction schemes may be varied to facilitate the reaction or to avoid unwanted reaction products.

Methods for preparing β-D-N(4)-hydroxycytidine (NHC), and derivatives thereof are disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety. Methods for preparing Compound A is disclosed in PCT International Patent Application No. PCT/US2021/048054, filed Aug. 27, 2021, as set forth below.

Synthesis of Uridine 4-oxime 5′-(2-methylpropanoate) (Compound A)

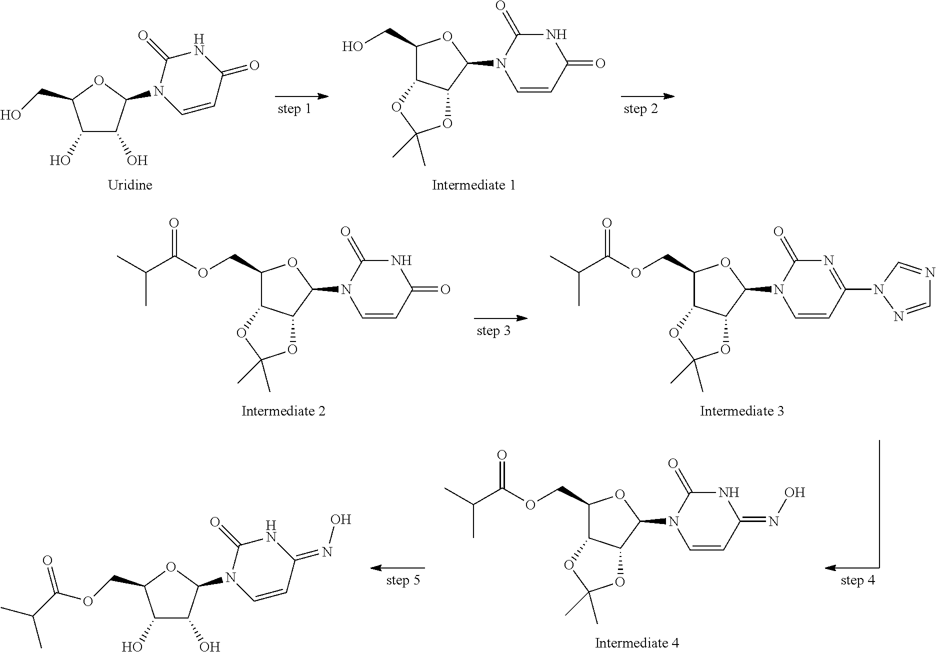 [SMILES: O=c1ccn([C@@H]2O[C@H](CO)[C@@H](O)[C@H]2O)c(=O)[nH]1 ; CC1(C)O[C@@H]2[C@H](O1)[C@@H](CO)O[C@H]2n1ccc(=O)[nH]c1=O ; CC(C)C(=O)OC[C@H]1O[C@@H](n2ccc(=O)[nH]c2=O)[C@@H]2OC(C)(C)O[C@@H]21 ; CC(C)C(=O)OC[C@H]1O[C@@H](n2ccc(-n3cncn3)nc2=O)[C@@H]2OC(C)(C)O[C@@H]21 ; CC(C)C(=O)OC[C@H]1O[C@@H](n2cc/c(=N/O)[nH]c2=O)[C@H](O)[C@@H]1O ; CC(C)C(=O)OC[C@H]1O[C@@H](n2cc/c(=N/O)[nH]c2=O)[C@@H]2OC(C)(C)O[C@@H]21]

A 100 L cylindrical vessel was charged with uridine (11.66 kg), acetone (70 L), 2,2-dimethoxypropane (1.05 equiv), and sulfuric acid (0.01 equiv). The reaction mixture was heated at 50° C.-57° C. until the reaction was deemed complete. Triethylamine (0.04 equiv) was added, followed by seed, and the slurry was cooled to 0° C.-5° C. The crystalline solid was collected and washed with MTBE to afford Intermediate 1.

Intermediate 1 was charged into a 100 L cylindrical vessel, followed by ethyl acetate (40 L), triethylamine (1.44 equiv), and DMAP (0.02 equiv). The mixture was cooled, and isobutyric acid was added slowly. The reaction mixture was aged at 20° C.-25° C. until full conversion was observed. The organic solution was washed twice with water, then azeotropically dried to afford a 29.4 wt % solution of Intermediate 2 in ethyl acetate.

In a 100 L cylindrical vessel was mixed 1,2,4-triazole (4.94 equiv), acetonitrile (36 L), triethylamine (6.88 equiv), and POCl₃ (1.28 equiv). A portion of the solution of Intermediate 2 (22.9 mol) was added to the vessel, and the resulting mixture was aged at ambient temperature overnight. Ethyl acetate was added, and the organic solution was washed twice with water. The solvent was exchanged for dry isopropanol by distillation, and heptane was added to crystallize Intermediate 3, which was collected by filtration and washed with MTBE.

A mixture of Intermediate 3 and acetonitrile (15 L) was cooled in a 100 L cylindrical vessel and reacted with aqueous hydroxylamine (1.3 equiv) until the reaction was deemed complete. Water was added, and the crystalline product was isolated by filtration and washed with water to afford Intermediate 4.

Intermediate 4 (5.96 kg) was added to a 100 L cylindrical vessel, along with acetonitrile (60 L) and aqueous HCl (1.27 equiv). The reaction was aged at 31° C.-33° C. until the reaction was deemed complete. The acid was quenched with aqueous sodium carbonate and the acetonitrile was replaced with ethyl acetate through distillation. The organic phase was washed with 22 wt % aqueous sodium sulfate and water. The resulting ethyl acetate solution was azeotropically dried to crystallize the product. MTBE was added, and the product was collected by filtration and washed with a mixture of ethyl acetate and MTBE to afford uridine 4-oxime 5′-(2-methylpropanoate) (Compound A).

Recrystallization of Uridine 4-oxime 5′-(2-methylpropanoate) (Compound A)

Compound A was recrystallized by dissolving Compound A, from the above synthesis in acetone, heating to 50° C., and allowing the solution to cool to room temperature and exposed to n-heptane vapors by vapor diffusion.

### Methods, Uses, and Medicaments

Products provided as therapies may include a composition comprising an antiviral nucleoside in a composition.

Non-limiting embodiments include:

1. A method of treating a viral infection in a subject in need thereof, said method comprising administering to the subject an antiviral nucleoside; wherein
the antiviral nucleoside is administered twice daily for 1 to 10 days;
the antiviral nucleoside is selected from Compound A:

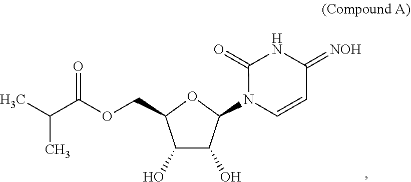

prodrugs of Compound A, tautomers of Compound A, pharmaceutically acceptable salts of Compound A, and mixtures thereof; and
the viral infection is an infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus.

2. The method according to embodiment 1, wherein the virus is selected from the group consisting of RSV, influenza A virus, influenza B virus, filoviridae virus, human coronavirus, SARS-CoV-1, MERS-CoV, SARS-CoV-2, and Ebola virus.

3. The method according to embodiment 1, wherein the virus is selected from the group consisting of influenza A virus and influenza B virus.

4. The method according to embodiment 1, wherein the virus is selected from the group consisting of human coronavirus, SARS-CoV-1, MERS-CoV, and SARS-CoV-2.

5. The method according to embodiment 1, wherein the virus is SARS-CoV-2.

6. The method of any one of embodiments 1 to 5, wherein the antiviral nucleoside is administered orally.

7. The method of any one of embodiments 1 to 6, wherein the antiviral nucleoside is administered twice daily, as individual doses in an amount of from about 200 mg to about 1200 mg.

8. The method of embodiment 7, wherein the antiviral nucleoside administered twice daily, as individual doses in an amount of about 800 mg.

9. The method of any one of embodiments 1 to 8, wherein the antiviral nucleoside is administered twice daily for 5 days.

10. The method of any one of embodiments 1 to 9, wherein said administering is initiated within 5 days of symptom onset.

11. The method of any one of embodiments 1 to 10, wherein the antiviral nucleoside is provided as individual doses using 1, 2, 3, or 4 200 mg capsules.

12. The method of embodiment 11, wherein the antiviral nucleoside is provided as individual doses using 4 200 mg capsules.

13. The method of any one of embodiments 1 to 12, wherein said method results in a reduction in risk of hospitalization or death for the subject.

14. The method of embodiment 13, wherein said method results in a reduction in risk of hospitalization or death for the subject of 1 to 10 percent.

15. The method of embodiment 13, wherein said method results in a relative reduction in risk of hospitalization or death for the subject of up to about 50 percent.

16. The method of any one of embodiments 1 to 15, wherein said subject is determined to be at increased risk for severe illness from COVID-19.

17. The method of embodiment 16, wherein said subject has one or more underlying medical condition associated with being at increased risk for severe illness from COVID-19.

18. A method of treating SARS-CoV-2 in a subject in need thereof, comprising administering {(2R,3S,4R,5R)-3,4-dihydroxy-5-[(4Z)-4-(hydroxyimino)-2-oxo-3,4-dihydropyrimidin-1(2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days.

19. A method of reducing the risk of hospitalization or death due to SARS-CoV-2 in a subject in need thereof, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the risk of hospitalization or death due to SARS-CoV-2 is reduced compared to risk of hospitalization or death due to SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

20. A method of reducing time to SARS-CoV-2 RNA clearance in a subject in need thereof, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the time to SARS-CoV-2 RNA clearance is reduced compared to time to viral clearance in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

21. A method of reducing SARS-CoV-2 viral load in a subject in need thereof, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the viral load of SARS-CoV-2 is reduced compared to viral load in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof,.

22. A method of reducing SARS-CoV-2 infectious virus in a subject in need thereof, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl} methyl 2-methylpropanoate, prodrugs of Compound A, tautomers of Compound A, pharmaceutically acceptable salts of Compound A, or mixtures thereof, to the subject twice daily for 1 to 10 days,
wherein the infectious virus is reduced compared to infectious virus in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

23. A method of reducing morbidity or mortality due to SARS-CoV-2 in a subject in need thereof, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the morbidity or mortality due to SARS-CoV-2 is reduced compared to morbidity or mortality due to SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

24. A method of reducing time to normalization of high-sensitivity C-reactive protein in a subject with SARS-CoV-2, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the time to normalization of high-sensitivity C-reactive protein in a subject with SARS-CoV-2 is reduced compared to time to normalization of high-sensitivity C-reactive protein in a subject with SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

25. A method of reducing time to normalization of oxygen saturation in a subject with SARS-CoV-2, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the time to normalization of high-sensitivity C-reactive protein and of oxygen saturation due to SARS-CoV-2 is reduced compared to time to normalization of high-sensitivity C-reactive protein and of oxygen saturation in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

26. A method of reducing need for respiratory interventions in a subject with SARS-CoV-2, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, to the subject twice daily for 1 to 10 days,
wherein the need for respiratory interventions in a subject with SARS-CoV-2 is reduced compared to need for respiratory interventions in a subject with SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

27. A method of reducing length of hospitalization in a subject with SARS-CoV-2, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate to the subject twice daily for 1 to 10 days,
wherein the length of hospitalization in a subject with SARS-CoV-2 is reduced compared to length of hospitalization in a subject with SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof.

28. The method of any one of embodiments 18-27, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, is administered to the subject orally.

29. The method of any one of embodiments 18-28, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, is administered to the subject twice daily, as individual doses in an amount of from about 200 mg to about 1200 mg (2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

30. The method of embodiment 29, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is administered twice daily, as individual doses of about 800 mg.

31. The method of any one of embodiments 18 to 30, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, is administered twice daily for 5 days.

32. The method of any one of embodiments 18 to 31, wherein administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, is initiated within 5 days of symptom onset.

33. The method of any one of embodiments 18 to 32, wherein administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate, prodrugs of Compound A, tautomers of Compound A, pharmaceutically acceptable salts of Compound A, or mixtures thereof, is initiated within 5 days of confirmation of SARS-CoV-2 infection.

34. The method of any one of embodiments 18 to 33, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is provided as individual doses using 1, 2, 3, or 4 200 mg capsules.

35. The method of embodiment 34, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is provided as individual doses using 4 200 mg capsules.

36. The method of any one of embodiments 18 to 35, wherein said method results in a reduction in risk of hospitalization or death for the subject.

37. The method of embodiment 36, wherein said method results in a reduction in risk of hospitalization or death for the subject of from 1 to 10 percent.

38. The method of embodiment 36, wherein said method results in a relative reduction in risk of hospitalization or death for the subject of up to about 50 percent.

39. The method of one of embodiments 18 to 38, wherein said subject has at least one risk factor for development of severe COVID-19.

40. The method of embodiment 39, wherein the at least one risk factor is chosen from age (60, older than 60, 65, or older than 65), active cancer,chronic kidney disease, chronic obstructive pulmonary disease, obesity (e.g., BMI ≥30), serious heart conditions (e.g., heart failure, coronary artery disease, or cardiomyopathies), and diabetes mellitus.

41. The method of any one of embodiments 18 to 40, wherein the subject does not have at least one exclusion criterion chosen from anticipated need for hospitalization for Covid-19 within the next 48 hours, dialysis or estimated glomerular filtration rate less than 30 ml per minute per 1.73 m2, pregnancy, unwillingness to use contraception during the intervention period and for at least 4 days after completion of the regimen, severe neutropenia (absolute neutrophil count of <500 per milliliter), platelet count below 100,000 per microliter, and SARS-CoV-2 vaccination.

In one embodiment, this disclosure provides an antiviral nucleoside for use in therapy. In one embodiment, the therapy is the treatment of a viral infection, such as infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In aspects of this embodiment, the virus is selected from the group consisting of RSV, influenza A virus, influenza B virus, filoviridae virus, human coronavirus, SARS-CoV-1, MERS-CoV, SARS-CoV-2, and Ebola virus. In more specific aspects, the virus is selected from the group consisting of influenza A virus and influenza B virus. In other specific aspects, the virus is selected from the group consisting of human coronavirus, SARS-CoV-1, MERS-CoV, and SARS-CoV-2. In even more specific aspects, the virus is SARS-CoV-2. In particular aspects, the virus is SARS-CoV-2 and various clades thereof. In even more particular aspects, the virus is selected from clades of SARS-CoV-2.

The therapy may also comprise one or more additional therapeutic agents. The one or more additional active agents may be administered with antiviral nucleoside (co-administered) or administered separately from the antiviral nucleoside, in a different dosage form. That is, the additional active agent(s) may be administered in a single dosage form with the antiviral nucleoside, or the additional active agent(s) may be administered in separate dosage form(s) from the dosage form containing the antiviral nucleoside.

The therapies disclosed herein may be used in combination with one or more other active agents, including but not limited to, antiviral agents that are used in the prevention, treatment, control, amelioration, or reduction of risk of a particular disease or condition (e.g., viral infection). In one embodiment, a compound disclosed herein is combined with one or more other antiviral agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present disclosure.

When the therapies disclosed herein are used contemporaneously with one or more other active agents, the antiviral nucleoside may be administered either simultaneously with, or before or after, one or more other active agent(s). The antiviral nucleoside may be administered separately, by the same or different route of administration, or together in the same pharmaceutical composition as the other agent(s).

The dosage amount of the antiviral nucleoside may be varied and will depend upon the therapeutically effective dose of each agent. Generally, a therapeutically effective dose of each will be used. Combinations including at least one antiviral nucleoside, and other active agents will generally include a therapeutically effective dose of each active agent. In such combinations, the antiviral nucleosides disclosed herein and other active agents may be administered separately or in conjunction. In addition, the administration of one element may be prior to, concurrent with, or subsequent to the administration of other agent(s).

In one embodiment, this disclosure provides an antiviral nucleoside, and at least one other active agent as a combined preparation for simultaneous, separate or sequential use in therapy. In one embodiment, the therapy is the treatment of a viral infection, such as infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In aspects of this embodiment, the virus is selected from the group consisting of RSV, influenza A virus, influenza B virus, filoviridae virus, human coronavirus, SARS-CoV-1, MERS-CoV, SARS-CoV-2, and Ebola virus. In more specific aspects, the virus is selected from the group consisting of influenza A virus and influenza B virus. In other specific aspects, the virus is selected from the group consisting of human coronavirus, SARS-CoV-1, MERS-CoV, and SARS-CoV-2. In even more specific aspects, the virus is SARS-CoV-2.

In another embodiment, this disclosure provides an antiviral nucleoside, and at least one other active agent as a combined preparation for simultaneous, separate or sequential use in therapy. In one embodiment, the therapy is antiviral prophylaxis, such as for potential infection, either pre-exposure or post-exposure, by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In aspects of this embodiment, the virus is selected from the group consisting of RSV, influenza A virus, influenza B virus, filoviridae virus, human coronavirus, SARS-CoV-1, MERS-CoV, SARS-CoV-2, and Ebola virus. In more specific aspects, the virus is selected from the group consisting of influenza A virus and influenza B virus. In other specific aspects, the virus is selected from the group consisting of human coronavirus, SARS-COV-1, MERS-CoV, and SARS-CoV-2. In even more specific aspects, the virus is SARS-CoV-2.

The disclosure also provides the use of an antiviral nucleoside for treating a viral infection, where the patient has previously (e.g., within 24 hours) been treated with another agent.

The additional active agent(s) may be one or more agents selected from the group consisting of antiviral compounds, antigens, adjuvants, anti-cancer agents, CTLA-4 agonists, LAG-3 agonists, PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, antibiotics, anti-metabolites, retinoids, steroids, and immunomodulatory agents, including but not limited to antiviral vaccines. It will be understood the descriptions of the above additional active agents, and of those listed below, may be overlapping. It will also be understood that the treatment combinations are subject to optimization, and it is understood that the best combination to use of the antiviral nucleoside, and one or more additional active agents will be determined based on the individual patient needs.

Antiviral compounds that may be used in combination with the therapies disclosed herein include direct acting antivirals and antiviral compounds that target intracellular environments. In particular, antiviral compounds that may be used in combination with the therapies disclosed herein include antivirals that target SARS-CoV-2 virus (and COVID-19 caused by SARS-CoV-2 infection), influenza, hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors. Such antiviral compounds include but are not limited to 2-DG, 2x-121, AB001, avifavir, AVM-0703, C21, CAL-02, CYTO-201 (naltrexone hydrochloride), Conronavir (TL-FVP-t), DW-2008S, DWJ-1248, elsufavirine, emtricitabine, eFT226, HP-163, IML-206, IMU-838, LAU-7b, MAN-19, MMS-019, OBP-2001, omega 3 viruxide, OPN-019, OYA-1, PP-001, PRTX-007, RBI-5000, RBT-9, RECCE529, RS-5614, SK11, SLV-213, T-COVID, TL-895, TYME-19, UCI-1, XC-221, fenretinide, nafamostat, nafamostat mesylate, nanomedivir (atazanavir/dexamethasone), nanofenretinide (ST-001), necuparanib (M-402), nelfinavir, nitazoxanide, piclidenoson, pixatimod, polyinosinic-plycytidylic acid, proxalutamide, hydrochloroquine, hydroxychloroquine, chloroquine, oseltamivir, oseltamivir phosphate, zanamivir, peramivir, baloxavir marboxil, remdesivir, favilavir, avifavir, favilavir/avifavir, vaniprevir, grazoprevir, elbasvir, narlaprevir, nitazoxanide, atazanavir, ritonavir, daclatasvir, farunavir, darunavir/cobicistat, saquinavir, indinavir, carfilzomib, ivaltinostat (CG200745), isotretinoin/tamoxifen, isotretinoin, tamoxifen, levamisole, prexasertib, ebselen, merimepodib, 1-deoxy-D-glucose prodrugs, formoterol, budesonide, rigosertib, erlotinib, silmitasertib, favipiravir, galidesivir, ledipasvir, lopinavir, lopinavir/ritonavir, levovir, tenofovir, and sofosbuvir, and combinations thereof.

Additional therapies that may be used in combination with the therapies disclosed herein include but are not limited to immunomodulators, such as interleukin 6 (IL-6) inhibitors, corticosteroids, TNF-inhibitors, and other immune-dependent therapies; antibody therapies, such as convalescent plasma therapies, hyperimmune globulin therapies, monoclonal antibodies, polyclonal antibodies, and neutralizing antibodies; soluble guanylate cyclase stimulator, such as riociguat; cannibidiols; and vaccines. The additional therapies contemplated include biological products that are biosimilar to any biological product or therapy expressly listed herein.

In particular, the additional therapies that may be used in combination with the therapies disclosed herein include but are not limited to 2,3,4,5,6-pentafluoro-N-(3-fluoro-4-methoxyphenyl)benzene sulfonamide, 3′,4′-didehydro-4′deoxy-8′-norvin-caleukoblastine, 47D11, 5-fluorouracil, abatacept, abiraterone acetate, ABX464, abibertinib, acalabrutinib, ACE2-Fc, ACE-MAB (STI-4920, CMAB020), acetylsalicylic acid, acetaminophen, ACT-20, Actemra, Actemra/RoActemra, adalimumab, adipose mesenchymal cells, AdMSCs (autologous adipose-derived stem cells), ADR-001, adrecizumab (HAM8101), ADX-629/reproxalap, AK-119, Alferon N, Allocetra (leukocyte cell based therapy), AlloStim, Allorx stem cells, ALT-100 (enamptcumab), ALT-803, Amnioboost, Ampion, altretamine, amiodarone, Anaferon, Anakinra, AMG-3777, anhydrovinblastine, anti-nCoV nanoviricides, aprepitant, AP-003 (AntiCovir), APL-9 (pegylated synthetic cyclic peptide), APX-115, AQCH, AR-701, ARO-COV, AS-1411, ascorbic acid, asunercept, atovaquone/azithromycin, AT-100 (rhSP-D), AT-301, AT-H201, ATI-450, ATR-002, auristatin, avdoralimab (IPH5401), axatilimab, AZD-1061, AZD-7442, alvelestat (AZD-9668), AZD-8895, azvudine, azvudine/tetrandrine, azithromycin, bardoxolone, bardoxolone methyl, baricitinib, BBT-032, bemcentinib, BGE-175, BIO-300, BIOMEDIVR, bevacizumab, bexarotene, bicalutamide, BIO-1106, BLD-2660, BLD-2736, BOLD-100, brequinar sodium, brilacidin, bromhexine hydrochloride, BTL-TML001, bleomycin, BMS-986253, BMS 184476, BT-086, BT-588, BXCL501, BXT-25, bucillamine, budesonide, cachectin, acalabrutinib, camrelizumab, camrelizumab/thymosin, captopril, CardioIRx, carrimycin, cavaltinib, comostat, camostat mesylate, canakinumab, CAP-1002, carboplatin, carmustine, CB5064 analogs, CD24Fc (recombinant fusion protein), cepharanthine, cemadotin, cenicriviroc, canthaquine, CERC-002, chlorambucil, chloropromazine, cholecalciferol, ciclesonide, cisplatin, ci-trimoxazole, CK-0802, clazakizumab, clarithromycin, CLBS-119, CM4620-IE, colchicine, CorLiCyte (umbilical cord lining stem cells), COVID-19 aptamer therapy, COVID-19 human mAb, COVID-19 neutralizing antibodies, COVID-19 siRNA therapy, COVID-HIG, COVID-EIG, spike glycoproteins, CoviGlobulin, COVI-GUARD (STI-1499), CPI-006, crizanlizumab, cryptophycin, CSL-324, CT-P59, CTAP-101, CV-15, CVL-218, cyclosporine, cell replacement therapies, cyclophosphamide, CYNK-001, cytarabine, dacarbazine, dactolisib, dactinomycin, dalargin, DAS-181, dapagliflozin, dapansurtrile, daunorubicin, decitabine, dexamethasone, DNL758 (SAR443122, RIPK1 inhibitor), dipyridamole, DMX-200, DS-2319, deupirfenidone, duvelisib, DV-890, DWRX-2003, docetaxol, dolastatin, doxetaxel, doxorubicin (adriamycin), DP-710, EB-05, EB-201, ebastine, eculizumab, EDP-1815, efineptakin alfa, emapalumab, emtricitabine, ensifontrine, ENU-200, enoxaparin, enzalutamide, epaspire, etanercept, etoposide, eravacycline, famotidine, finasteride, fingolimod, flebogamma (IGIV31), fluvoxamine, foalumab (NI-0401, TZLS-401), fostamatinib, flutamide, FSD-201, FW1022, FT516, Gamunex (IGIV-C), ganetespib, GC-376, Giapreza, GLS-1200, garadacimab, GC-5131A (hyperimmune globulin), GIGA-2050 rCIG), gimsilumab, GNS561, GP1681, GSK-2586881/APN-1, GSK-4182136, GTB-3550 (Trike 161533), haNK: CD-16, HB-adMSCs, HFB30132A, HLCM-051, heparin, hydrocortisone, hydroxyurea, ibuprofen, ibudilast (MN-166), icosapent ethyl, IC14, IDB-003, IFX-1/BDB-1, IgY-110, IMM101 IMS001, IMS002, ifosfamide, imatinib, infliximab, INM-005, interferon alfa, interferon alfa 1B, interferon alfa 2B, interferon beta 1A, interferon beta 1B, interleukin-6, interleukin-7, isoquercetin, itanapraced (CHF-5074), itolizumab, ivermectin, IVIG, JS012 (monoclonal antibody, LY-CoV016), jaktinib, kagocel, KB109, sarilumab, K-NK-ID101, KTH-222, lactoferrin, LAM-002A (apilimod dimesylate), lanadelumab, lamellasome, LB-1148, larazotide, leflunomide, lenzilumab, leronlimab (monoclonal antibody), levilimab (BCD-089), levamisole, liarozole, linagliptin, lipocure, losartan, livilimab, lomustine (CCNU), lonidamine, losmapimod, lostartan, LY-CoV555 (LY-3819253), LY-3127804, mannitol, maraviroc, mastinib, mavrilimumab, MDV3100, mechlorethamine, MEDI-3506, melatonin, melphalan, meplazumab, merimepodib, Mesenchymal stem cells (MSCs), mesencure (cell replacement), metablok (anti-inflammatory), metformin, methotrexate, methylprednisolone, mitomycin, mivobulin isethionate, mosedipimod (EC-18), MP-0420, MP-0423, MRx4DP0004, N-acetylcysteine, N,N-dimethyl-L-valyl-L-valyl-N-methyl-L-valyl-L-prolyl-1-Lproline-t-butylamide, namilumab (IZN-101), nangibotide, narsoplimab, nebulized dornase alfa, NED-260, Niagen (nicotinamide riboside; Vitamin B3), NK cell therapy, niclosamide, nilutamide, nintedanib, nitric oxide, nivolumab, NL-CVX1, NLP-21, NP-02, N-120 (ifenprodil), novaferon, NT-17 (efineptakin alfa), NTR-441, octagam, olokizumab, omeprazole, onapristone, opaganib, OP-101, OT-101 (trabedersen), otilimab, ozanimod, paclitaxel, pacritinib, panaphix, pamrevlumab, paracetamol, PB1046, PTC299, pegylated interferon alpha, pegylated interferon alpha 2b, pegylated interferon lambda, pembrolizumab, PL-8177, pirfenidone, plitidepsin (aplidin), PneumoBlast, polyoxidonium, prazosin, prednimustine, prednisolone, prednisone, pritumumab, procarbazine, prolastin, PTC-299, pyronaridine/artesunate, radotinib, RAPA-501, ravulizumab, razuprotafib, interferon beta 1 agonists, RECC327, REGN-COV2 (antibody cocktail), reparixin, rintatolimod (ampligen), RLF-100 (aviptadil), RLS-0071, STI-5656 (abivertinib), Rhu-pGSN (gelsolin), rhizoxin, RPR109881, RoActemra, RUCONEST (conestat alfa), ruxolitinib, SAB-185, SAR443122, SARS-CoV-2 antibodies, SARS-CoV-2 monoclonal antibodies, SARS-CoV-2 polyclonal antibodies, SARS-COV-2 neutralizing antibodies, SCTA01, Leukine (sargramostim), selenexor, sevoflurane, sertenef, siltuximab, sildenafil citate, silymarin, simvastatin, sirolimus, sirukumab, SIWA-318, solnatide, SNG-001, ST-266, stem cell educator therapy, STI-1499, STI-2020dna (COVI-MAB), STI-4398 (Covidtrap), stramustine phosphate, streptozocin, T cell therapies (TargNaturTa), TAK-671, TAK-888, TATX-36, TATX-99, TCB-007, TJ003234/TJM-2, TP508, TRV027, TD-0903, TLC19, tekrurna, tafenoquine, tamoxifen, tasonermin, taxanes, taxol, tetradrine, thalidomide, thimerosal, thymalfasin, tinzaparin, tocilizumab, tofacitinib, toremifene, tradipitant, tranexamic acid, trans sodium crocetinate (TSC), tramadol, tretinoin, TXA127 (antiotensin-(1-7) peptide), TY027, TZLS-501, UNI-911, ulinastatin, upamostat, vafidemstat, valsartan, icosapent ethyl, vazegepant, VBI-S, VERU-111, VHH72-Fc, vinblastine, vincristine, vindesine sulfate, vinflunine, VIR-2703 ALN-COV), VIR-7831, VIR-7832, Vitamin C, Vitamin D, XAV-19, Xpro-1595, XRx-101, zanubrutinib, zilucoplan, and zinc, and combinations thereof.

The additional vaccine therapies that may be used in combination with the therapies disclosed herein include but are not limited to inactivated vaccines, live-attenuated vaccines, recombinant vaccines, replication-deficient viral vector vaccines, mRNA-based vaccines, DNA vaccines, nanoparticle vaccines, non-replicating viral vectors, self-replicating RNA vaccines, self-amplifying RNA vaccines, protein subunit vaccines, Ii-Key peptide COVID-19 vaccines, gp96-based vaccines, intranasal vaccines, and mRNA lipid nanoparticle (mRNA-LNP) vaccine. In particular, the additional vaccine therapies that may be used in combination with the therapies disclosed herein include but are not limited to 7HP-349, AAVCOVID (gene-based vaccine), Ad26.COV2-S(non-replicating viral vector), Ad5-nCoV (recombinant vaccine; adenovirus type 5 vector), Ad5-S-nb2, AdCOVID (intranasal vaccine), AdimrSC-2F (protein subunit vaccine), AG0301-COVID19 (DNA vaccine), AKS-446, ARCoV, AV-COVID-19, AVI-205, AZD1222 (replication-deficient viral vector vaccine (adenovirus from chimpanzees)), Bacillus Calmette-Guerin (BCG) vaccine (live attenuated vaccines), bacTRL-Spike (monovalent oral vaccine (bifidobacterial)), BBIBP-CorV (inactivated vaccine), BC-PIC COVID-19 vaccine, BNT-162 (mRNA-based vaccine), BVX-0320, CDX-005, ChAd-SARS-CoV-2-S(adenovirus-based vaccine, Chimigen vaccine, CIGB-2020, CiVax, Coravax, CoroFlu, COV001/AZD-1222), CoronaVac (inactivated vaccine (formalin with alum adjuvant)), Corvax, CORVax-12, COVAX-19 (monovalent recombinant protein vaccine), Covaxin (BBV-152, inactivated vaccine), CoVepiT, CVnCoV (mRNA-based vaccine), DPX-COVID-19, DS-5670, E-6020, ELI-005, EpiVacCorona, EPV-CoV19, Flowvax, GC-004/Covax-19, GRAd-COV2 (adenovirus-based vaccine), GX-19 (DNA vaccine), HaloVax (self-assembling vaccine), HDT-301 (RNA vaccine), iBIO-201, IK-15800, INO-4700, INO-4800 (DNA vaccine (plasmid)), IPT-001, ISR-50, KBP-COVID-19, LEAPS COVID-19 vaccine, LineaDNA (DNA vaccine), LNP-mRNA, LNP-nCoVsaRNA, LUNAR-COVV19 (ARCT-021; self-replicating RNA vaccine), mRNA-1273 (mRNA-based vaccine), MAPS vaccine, MT-2766, MV-014-210, MVA-S, MVC-COV-1901, NVX-CoV233 (nanoparticle vaccine), NVX-CoV2373, Oncoquest, OraCOV, PDS-0203, PDS-0204, PiCoVacc, PittCoVacc (Recombinant protein subunit vaccine (delivered through microneedle array)), PolyPEPI-SCoV-2, QAZCOVID-IN, repRNA-CoV2S (LION/repRNA-CoV2S), RNAi vaccine, RV-1730m, rVSV COVID-19 vaccine, S-268019, Saponin vaccine adjuvant, SCB-2019 (protein subunit vaccine), Shingrix vaccine (GSK-1437173A), Sputnik-V (Gam-COVID-Vac), STI-6991, T-COVID™ (intranasal vaccine), TaliCoVax-19, TerraCoV2, Tertomotide (GV-1001), Tiba-pitt RNA vaccine, TNX-1800, TNX-1810, TNX-1820, TNX-2300, T-VIVA-19, V-590 (recombinant vaccine (vesicular stomatitis virus)), V-591 (measles vector vaccine), VBI-2900, VBI-2901, VBI-2902, VLA2001, Vivagel (SPL-7013), YF17D vector, ZIP-1642, and ZyCoV-D (DNA vaccine (plasmid)), and combinations thereof.

Additionally, the present disclosure relates to methods of treating a viral infection, wherein the method comprises administering to a subject in need thereof a therapy that comprises an antiviral nucleoside; wherein the viral infection is an infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of the antiviral treatment methods disclosed herein, the antiviral nucleoside is selected from forms of Compound A. In particular embodiments of the antiviral treatment methods disclosed herein, the antiviral nucleoside is Compound A.

While not wanting to be bound by a single theory, as currently understood, the mechanism of antiviral activity of Compound A is viral error catastrophe predicated on increasing the viral mutation rate beyond a biologically-tolerable threshold resulting in impairment of viral fitness leading to viral extinction. This proposed mechanism of action involves tautomerization and different base-pairing interactions, as illustrated below (circles represent ribosyl subunits).

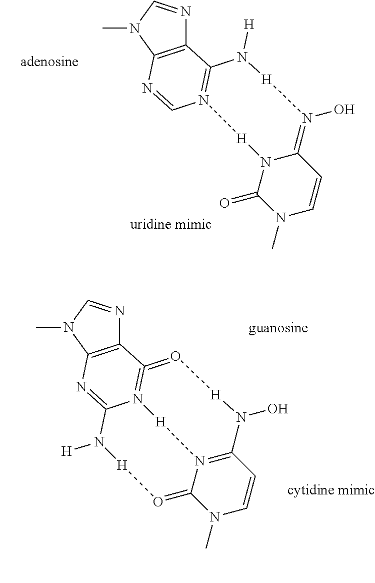

In embodiments of the treatment methods disclosed herein, the antiviral nucleoside is administered orally, by intravenous infusion, or by subcutaneous injection. In specific embodiments, the antiviral nucleoside is administered orally.

In embodiments of the treatment methods disclosed herein, the antiviral nucleoside is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered once daily, as a single dose of an amount of about 200 mg. In aspects of such embodiments, the antiviral nucleoside is administered once daily, as a single dose of an amount of about 400 mg. In aspects of such embodiments, the antiviral nucleoside is administered once daily, as a single dose of an amount of about 600 mg. In aspects of such embodiments, the antiviral nucleoside once daily, as a single dose of administered at an amount of about 800 mg. In aspects of such embodiments, the antiviral nucleoside is administered once daily, as a single dose of an amount of about 1000 mg. In aspects of such embodiments, the antiviral nucleoside is administered once daily, as a single dose of an amount of about 1200 mg. In aspects of such embodiments, the antiviral nucleoside is administered once daily, as a single dose of an amount of about 1400 mg. In aspects of such embodiments, the antiviral nucleoside is administered once daily, as a single dose of an amount of about 1600 mg. In particular aspects of these embodiments, the antiviral nucleoside may be provided as individual doses using 1, 2, 3, 4, 5, 6, 7, or 8 200 mg capsules as individual unit dosage forms. In more specific embodiments, the antiviral nucleoside may be provided as individual doses using 4 200 mg capsules as individual unit dosage forms. In other specific embodiments, the antiviral nucleoside may be provided as individual doses using 8 200 mg capsules as individual unit dosage forms. In specific embodiments, the individual unit dosage forms are 200 mg capsules.

In embodiments of the treatment methods disclosed herein, the antiviral nucleoside is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered twice daily, as individual doses of an amount of about 50 mg, about 100 mg, about 200 mg, about 300 mg, about 400 mg, about 500 mg, about 600 mg, about 700 mg, about 800 mg, or about 1000 mg. In aspects of such embodiments, the antiviral nucleoside is administered twice daily, as individual doses of an amount of about 200 mg. In aspects of such embodiments, the antiviral nucleoside is administered twice daily, as individual doses of an amount of about 400 mg. In aspects of such embodiments, the antiviral nucleoside is administered twice daily, as individual doses of an amount of about 600 mg. In aspects of such embodiments, the antiviral nucleoside is administered twice daily, as individual doses an amount of about 800 mg. In aspects of such embodiments, the antiviral nucleoside is administered twice daily, as individual doses of an amount of about 1000 mg. In particular aspects of these embodiments, the antiviral nucleoside may be provided as individual doses using 1, 2, 3, 4 or 5 200 mg capsules as individual unit dosage forms. In more specific embodiments, the antiviral nucleoside may be provided as individual doses using 4 200 mg capsules as individual unit dosage forms.

In embodiments of the treatment methods disclosed herein, said method comprises administering to a subject in need thereof a therapy that comprises an antiviral nucleoside; wherein the antiviral nucleoside is administered once daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the antiviral nucleoside is administered once daily for 3 to 14 days, such as for 3 days, 4 days, 5 days, 6 days, 7 days, 8 days, 9 days, 10 days, 11 days, 12 days, 13 days, or 14 days. In specific embodiments, the antiviral nucleoside is administered once daily for 3 to 6 days, such as for 3 days, 4 days, 5 days, or 6 days. In another specific embodiment, the antiviral nucleoside is administered once daily for 5 days.

In embodiments of the treatment methods disclosed herein, said method comprises administering to a subject in need thereof a therapy that comprises an antiviral nucleoside; wherein the antiviral nucleoside is administered twice daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the antiviral nucleoside is administered twice daily for 3 to 14 days, such as for 3 days, 4 days, 5 days, 6 days, 7 days, 8 days, 9 days, 10 days, 11 days, 12 days, 13 days, or 14 days. In specific embodiments, the antiviral nucleoside is administered twice daily for 3 to 6 days, such as for 3 days, 4 days, 5 days, or 6 days. In another specific embodiment, the antiviral nucleoside is administered twice daily for 5 days.

The disclosure further relates to a method of treating a viral infection, said method comprising administering to a subject in need thereof a therapy that comprises an antiviral nucleoside; wherein the therapy commences from 1 to 10 days after onset of symptoms of viral infection, such as 1 day, 2 days, 3 days, 4 days, 5 days, 6 days, 7 days, 8 days, 9 days, or 10 days after the onset of symptoms of viral infection. In specific embodiments, the antiviral nucleoside is administered beginning less than 5 days after onset of symptoms, such as less than 1 day, 2 days, 3 days, 4 days, or 5 days after onset of symptoms of viral infection. Symptoms of viral infection may include one or more of cough, sore throat, nasal congestion, runny nose, shortness of breath or difficulty breathing, muscle or body aches, fatigue/tiredness, feeling hot/feverish, chills, headache, nausea, vomiting, and diarrhea, although symptoms may vary by the type and severity of the viral infection. For example, symptoms of COVID-19 caused by SARS-CoV-2 viral infection may include one or more of cough, sore throat, nasal congestion, runny nose, shortness of breath or difficulty breathing, muscle or body aches, fatigue/tiredness, feeling hot/feverish, chills, headache, nausea, vomiting, diarrhea, loss of taste, and loss of smell.

The disclosure further relates to methods of treating a viral infection, wherein said subjects may be considered or is determined to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure further relates to methods of treating a viral infection, wherein said method reduces the risk of hospitalization or death for the subject. In embodiments, the method may result in a reduction in risk of hospitalization or death for the subject. In specific embodiments, the method may result in a reduction in risk of hospitalization or death for the subject of about 1 to about 10 percent, such as from about 5 to about 7.5 percent, or about 6.8 percent. In further embodiments, the method may result in a relative reduction in risk of hospitalization or death for the subject of up to about 50 percent.

The disclosure further relates to methods of providing antiviral prophylaxis, said method comprising administering to a subject in need thereof a therapy that comprises an antiviral nucleoside; wherein the therapy commences prior to exposure to a viral infection or after a potential exposure to a viral infection. In specific embodiments, the antiviral nucleoside is administered beginning prior to potential exposure to viral infection. In other specific embodiments, the antiviral nucleoside is administered from 1 to 10 days after potential exposure to a viral infection, such as 1 day, 2 days, 3 days, 4 days, 5 days, 6 days, 7 days, 8 days, 9 days, or 10 days after potential exposure. In specific embodiments, the antiviral nucleoside is administered beginning less than 5 days after potential exposure, such as less than 1 day, 2 days, 3 days, 4 days, or 5 days after potential exposure.

The disclosure further relates to methods of providing antiviral prophylaxis, wherein said subjects may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

Additionally, present disclosure relates to methods of providing antiviral prophylaxis, wherein the method comprises administering to a subject in need thereof a therapy that comprises an antiviral nucleoside; wherein the virus is selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of the prophylaxis methods disclosed herein, the antiviral nucleoside is Compound A.

In embodiments of the prophylaxis methods disclosed herein, the antiviral nucleoside is administered orally, by intravenous infusion, or by subcutaneous injection. In specific embodiments, the antiviral nucleoside is administered orally.

In embodiments of the prophylaxis methods disclosed herein, the antiviral nucleoside is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered once daily, as a single dose of an amount of about 200 mg. In aspects of such embodiments, the antiviral nucleoside is administered once daily, as a single dose of an amount of about 300 mg. In aspects of such embodiments, the antiviral nucleoside is administered once daily, as a single dose of an amount of about 400 mg. In aspects of such embodiments, the antiviral nucleoside is administered once daily, as a single dose of an amount of about 500 mg. In aspects of such embodiments, the antiviral nucleoside is administered once daily, as a single dose of an amount of about 600 mg. In aspects of such embodiments, the antiviral nucleoside is administered once daily, as a single dose of an amount of about 700 mg. In aspects of such embodiments, the antiviral nucleoside once daily, as a single dose of administered at an amount of about 800 mg. In particular aspects of these embodiments, the antiviral nucleoside may be provided as individual doses using 1, 2, 3, or 4 200 mg capsules as individual unit dosage forms. In more specific embodiments, the antiviral nucleoside may be provided as individual doses using 4 200 mg capsules as individual unit dosage forms.

In embodiments of the prophylaxis methods disclosed herein, the antiviral nucleoside is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered twice daily, as individual doses of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered twice daily, as individual doses of an amount of about 200 mg. In aspects of such embodiments, the antiviral nucleoside is administered twice daily, as individual doses of an amount of about 300 mg. In aspects of such embodiments, the antiviral nucleoside is administered twice daily, as individual doses of an amount of about 400 mg. In aspects of such embodiments, the antiviral nucleoside is administered twice daily, as individual doses an amount of about 500 mg. In aspects of such embodiments, the antiviral nucleoside is administered twice daily, as individual doses of an amount of about 600 mg. In aspects of such embodiments, the antiviral nucleoside is administered twice daily, as individual doses of an amount of about 700 mg. In aspects of such embodiments, the antiviral nucleoside twice daily, as individual doses of administered at an amount of about 800 mg. In particular aspects of these embodiments, the antiviral nucleoside may be provided as individual doses using 1, 2, 3, or 4 200 mg capsules as individual unit dosage forms. In more specific embodiments, the antiviral nucleoside may be provided as individual doses using 4 200 mg capsules as individual unit dosage forms.

The disclosure further relates to methods of providing antiviral prophylaxis, said method comprising administering to a subject in need thereof a therapy that comprises an antiviral nucleoside; wherein the antiviral nucleoside is administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside is administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside is administered once daily for 3 to 14 days, such as for 3 days, 4 days, 5 days, 6 days, 7 days, 8 days, 9 days, 10 days, 11 days, 12 days, 13 days, or 14 days.

The disclosure further relates to methods of providing antiviral prophylaxis, said method comprising administering to a subject in need thereof a therapy that comprises an antiviral nucleoside; wherein the antiviral nucleoside is administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside is administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside is administered twice daily for 3 to 14 days, such as for 3 days, 4 days, 5 days, 6 days, 7 days, 8 days, 9 days, 10 days, 11 days, 12 days, 13 days, or 14 days.

The disclosure further relates to prophylaxis methods, wherein said method reduces the risk of hospitalization or death for the subject. In embodiments, the method may result in a reduction in risk of hospitalization or death for the subject. In specific embodiments, the method may result in a reduction in risk of hospitalization or death for the subject of about 1 to about 10 percent, such as from about 5 to about 7.5 percent, or about 6.8 percent. In further embodiments, the method may result in a relative reduction in risk of hospitalization or death for the subject of up to about 50 percent.

Embodiments provided by this disclosure also include an antiviral nucleoside for use as a medicament for the treatment of viral infection. In specific embodiments, the viral infection is an infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In certain embodiments, the antiviral nucleoside may be selected from the antiviral nucleosides as disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety. In embodiments, the antiviral nucleosides is Compound A. In particular embodiments, the antiviral nucleoside is a mixture of Compound A and tautomers thereof. In other particular embodiments, the antiviral nucleoside is a mixture of Compound A, pharmaceutically acceptable salts of Compound A, tautomers of Compound A, and pharmaceutically acceptable salts of tautomers of Compound A.

An additional aspect of this embodiment relates to a pharmaceutical composition for use as a medicament for the treatment of viral infection, said pharmaceutical composition comprising (a) Compound A or a pharmaceutically acceptable salt thereof; and (b) a pharmaceutically acceptable carrier. In aspects of such embodiments, said pharmaceutical composition comprising (a) a mixture of Compound A and tautomers thereof, or pharmaceutically acceptable salts thereof; and (b) a pharmaceutically acceptable carrier.

The disclosure also provides an antiviral nucleoside for use as a medicament for the treatment of viral infection, where the patient has previously (e.g., within 24 hours) been treated with another agent, which may be as described above.

In embodiments of an antiviral nucleoside for use as a medicament for the treatment of viral infection, the antiviral nucleoside is administered orally, by intravenous infusion, or by subcutaneous injection. In specific embodiments, the antiviral nucleoside is administered orally.

In embodiments of an antiviral nucleoside for use as a medicament for the treatment of viral infection, the antiviral nucleoside is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered once daily, as a single dose of an amount of about 200 mg. In aspects of such embodiments, the antiviral nucleoside is administered once daily, as a single dose of an amount of about 400 mg. In aspects of such embodiments, the antiviral nucleoside is administered once daily, as a single dose of an amount of about 600 mg. In aspects of such embodiments, the antiviral nucleoside once daily, as a single dose of administered at an amount of about 800 mg. In aspects of such embodiments, the antiviral nucleoside is administered once daily, as a single dose of an amount of about 1000 mg. In aspects of such embodiments, the antiviral nucleoside is administered once daily, as a single dose of an amount of about 1200 mg. In aspects of such embodiments, the antiviral nucleoside is administered once daily, as a single dose of an amount of about 1400 mg. In aspects of such embodiments, the antiviral nucleoside is administered once daily, as a single dose of an amount of about 1600 mg. In particular aspects of these embodiments, the antiviral nucleoside may be provided as individual doses using 1, 2, 3, 4, 5, 6, 7, or 8 200 mg capsules as individual unit dosage forms. In more specific embodiments, the antiviral nucleoside may be provided as individual doses using 4 200 mg capsules as individual unit dosage forms. In other specific embodiments, the antiviral nucleoside may be provided as individual doses using 8 200 mg capsules as individual unit dosage forms. In specific embodiments, the individual unit dosage forms are 200 mg capsules.

In embodiments of an antiviral nucleoside for use as a medicament for the treatment of viral infection, the antiviral nucleoside is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered twice daily, as individual doses of an amount of about 50 mg, about 100 mg, about 200 mg, about 300 mg, about 400 mg, about 500 mg, about 600 mg, about 700 mg, about 800 mg, or about 1000 mg. In aspects of such embodiments, the antiviral nucleoside is administered twice daily, as individual doses of an amount of about 200 mg. In aspects of such embodiments, the antiviral nucleoside is administered twice daily, as individual doses of an amount of about 400 mg. In aspects of such embodiments, the antiviral nucleoside is administered twice daily, as individual doses of an amount of about 600 mg. In aspects of such embodiments, the antiviral nucleoside is administered twice daily, as individual doses an amount of about 800 mg. In aspects of such embodiments, the antiviral nucleoside is administered twice daily, as individual doses of an amount of about 1000 mg. In particular aspects of these embodiments, the antiviral nucleoside may be provided as individual doses using 1, 2, 3, 4 or 5 200 mg capsules as individual unit dosage forms. In more specific embodiments, the antiviral nucleoside may be provided as individual doses using 4 200 mg capsules as individual unit dosage forms.

In embodiments of an antiviral nucleoside for use as a medicament for the treatment of viral infection, the antiviral nucleoside is administered once daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the antiviral nucleoside is administered once daily for 3 to 14 days, such as for 3 days, 4 days, 5 days, 6 days, 7 days, 8 days, 9 days, 10 days, 11 days, 12 days, 13 days, or 14 days. In specific embodiments, the antiviral nucleoside is administered once daily for 3 to 6 days, such as for 3 days, 4 days, 5 days, or 6 days. In another specific embodiment, the antiviral nucleoside is administered once daily for 5 days.

In embodiments of an antiviral nucleoside for use as a medicament for the treatment of viral infection, wherein the antiviral nucleoside is administered twice daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the antiviral nucleoside is administered twice daily for 3 to 14 days, such as for 3 days, 4 days, 5 days, 6 days, 7 days, 8 days, 9 days, 10 days, 11 days, 12 days, 13 days, or 14 days. In specific embodiments, the antiviral nucleoside is administered twice daily for 3 to 6 days, such as for 3 days, 4 days, 5 days, or 6 days. In another specific embodiment, the antiviral nucleoside is administered twice daily for 5 days.

The disclosure further relates to an antiviral nucleoside for use as a medicament for the treatment of viral infection, wherein administration of the antiviral nucleoside is commenced 1 to 10 days after onset of symptoms of viral infection, such as 1 day, 2 days, 3 days, 4 days, 5 days, 6 days, 7 days, 8 days, 9 days, or 10 days after the onset of symptoms of viral infection. In specific embodiments, the antiviral nucleoside is administered beginning less than 5 days after onset of symptoms, such as less than 1 day, 2 days, 3 days, 4 days, or 5 days after onset of symptoms of viral infection. Symptoms of viral infection may be as described above.

The disclosure further relates to an antiviral nucleoside for use as a medicament for the treatment of viral infection, wherein the antiviral nucleoside is administered to a subject who may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure further relates to an antiviral nucleoside for use as a medicament for the treatment of viral infection, wherein administration of the antiviral nucleoside reduces the risk of hospitalization or death for the subject. In embodiments, administration of the antiviral nucleoside may result in a reduction in risk of hospitalization or death for the subject. In specific embodiments, administration of the antiviral nucleoside may result in a reduction in risk of hospitalization or death for the subject of about 1 to about 10 percent, such as from about 5 to about 7.5 percent, or about 6.8 percent. In further embodiments, administration of the antiviral nucleoside may result in a relative reduction in risk of hospitalization or death for the subject of up to about 50 percent.

The present disclosure also relates to an antiviral nucleoside for use as a medicament for prevention of viral infection or providing antiviral prophylaxis, wherein the viral infection to be prevented is caused by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In certain embodiments, the antiviral nucleoside may be selected from the antiviral nucleosides as disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety. In embodiments, the antiviral nucleosides is Compound A. In particular embodiments, the antiviral nucleoside is a mixture of Compound A and tautomers thereof. In other particular embodiments, the antiviral nucleoside is a mixture of Compound A, pharmaceutically acceptable salts of Compound A, tautomers of Compound A, and pharmaceutically acceptable salts of tautomers of Compound A.

An additional aspect of this embodiment relates to a pharmaceutical composition for use as a medicament for the prevention of viral infection or providing antiviral prophylaxis, said pharmaceutical composition comprising (a) Compound A or a pharmaceutically acceptable salt thereof; and (b) a pharmaceutically acceptable carrier. In aspects of such embodiments, said pharmaceutical composition comprising (a) a mixture of Compound A and tautomers thereof, or pharmaceutically acceptable salts thereof; and (b) a pharmaceutically acceptable carrier.

An additional aspect of this embodiment relates to a pharmaceutical composition for use as a medicament for the prevention of viral infection, said pharmaceutical composition comprising (a) Compound A or a pharmaceutically acceptable salt thereof; and (b) a pharmaceutically acceptable carrier. In aspects of such embodiments, said pharmaceutical composition comprising (a) a mixture of Compound A and tautomers thereof, or pharmaceutically acceptable salts thereof; and (b) a pharmaceutically acceptable carrier.

The disclosure also provides an antiviral nucleoside for use as a medicament for the prevention of viral infection or providing antiviral prophylaxis, where the patient has previously (e.g., within 24 hours) been treated with another agent, which may be as described above.

In embodiments of an antiviral nucleoside for use as a medicament for the prevention of viral infection or providing antiviral prophylaxis, the antiviral nucleoside is administered orally, by intravenous infusion, or by subcutaneous injection. In specific embodiments, the antiviral nucleoside is administered orally.

The disclosure further relates to an antiviral nucleoside for use as a medicament for the prevention of viral infection or antiviral prophylaxis, wherein the therapy commences prior to exposure to a viral infection or after a potential exposure to a viral infection. In specific embodiments, the antiviral nucleoside is administered beginning prior to potential exposure to viral infection. In other specific embodiments, the antiviral nucleoside is administered from 1 to 10 days after potential exposure to a viral infection, such as 1 day, 2 days, 3 days, 4 days, 5 days, 6 days, 7 days, 8 days, 9 days, or 10 days after potential exposure. In specific embodiments, the antiviral nucleoside is administered beginning less than 5 days after potential exposure, such as less than 1 day, 2 days, 3 days, 4 days, or 5 days after potential exposure.

The disclosure further relates to an antiviral nucleoside for use as a medicament for the prevention of viral infection or antiviral prophylaxis, wherein the antiviral nucleoside is administered to a subject who may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

Additionally, present disclosure relates to an antiviral nucleoside for use as a medicament for the prevention of viral infection or antiviral prophylaxis, wherein the antiviral nucleoside is administered to a subject who may have been exposed to a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of the antiviral nucleoside for use as a medicament for the prevention of viral infection or antiviral prophylaxis, the antiviral nucleoside is administered orally, by intravenous infusion, or by subcutaneous injection. In specific embodiments, the antiviral nucleoside is administered orally.

In embodiments of the antiviral nucleoside for use as a medicament for the prevention of viral infection or antiviral prophylaxis, the antiviral nucleoside is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered once daily, as a single dose of an amount of about 200 mg. In aspects of such embodiments, the antiviral nucleoside is administered once daily, as a single dose of an amount of about 300 mg. In aspects of such embodiments, the antiviral nucleoside is administered once daily, as a single dose of an amount of about 400 mg. In aspects of such embodiments, the antiviral nucleoside is administered once daily, as a single dose of an amount of about 500 mg. In aspects of such embodiments, the antiviral nucleoside is administered once daily, as a single dose of an amount of about 600 mg. In aspects of such embodiments, the antiviral nucleoside is administered once daily, as a single dose of an amount of about 700 mg. In aspects of such embodiments, the antiviral nucleoside once daily, as a single dose of administered at an amount of about 800 mg. In particular aspects of these embodiments, the antiviral nucleoside may be provided as individual doses using 1, 2, 3, or 4 200 mg capsules as individual unit dosage forms. In more specific embodiments, the antiviral nucleoside may be provided as individual doses using 4 200 mg capsules as individual unit dosage forms.

In embodiments of the antiviral nucleoside for use as a medicament for the prevention of viral infection or antiviral prophylaxis, the antiviral nucleoside is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered twice daily, as individual doses of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered twice daily, as individual doses of an amount of about 200 mg. In aspects of such embodiments, the antiviral nucleoside is administered twice daily, as individual doses of an amount of about 300 mg. In aspects of such embodiments, the antiviral nucleoside is administered twice daily, as individual doses of an amount of about 400 mg. In aspects of such embodiments, the antiviral nucleoside is administered twice daily, as individual doses an amount of about 500 mg. In aspects of such embodiments, the antiviral nucleoside is administered twice daily, as individual doses of an amount of about 600 mg. In aspects of such embodiments, the antiviral nucleoside is administered twice daily, as individual doses of an amount of about 700 mg. In aspects of such embodiments, the antiviral nucleoside twice daily, as individual doses of administered at an amount of about 800 mg. In particular aspects of these embodiments, the antiviral nucleoside may be provided as individual doses using 1, 2, 3, or 4 200 mg capsules as individual unit dosage forms. In more specific embodiments, the antiviral nucleoside may be provided as individual doses using 4 200 mg capsules as individual unit dosage forms.

The disclosure further relates to antiviral nucleoside for use as a medicament for the prevention of viral infection or antiviral prophylaxis, wherein the antiviral nucleoside is administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside is administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside is administered once daily for 3 to 14 days, such as for 3 days, 4 days, 5 days, 6 days, 7 days, 8 days, 9 days, 10 days, 11 days, 12 days, 13 days, or 14 days.

The disclosure further relates to antiviral nucleoside for use as a medicament for the prevention of viral infection or antiviral prophylaxis, wherein the antiviral nucleoside is administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside is administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside is administered twice daily for 3 to 14 days, such as for 3 days, 4 days, 5 days, 6 days, 7 days, 8 days, 9 days, 10 days, 11 days, 12 days, 13 days, or 14 days.

Embodiments provided by this disclosure also include uses of an antiviral compound in the preparation of a medicament for the treatment of viral infection. In specific embodiments, the viral infection is an infection by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In certain embodiments, the antiviral nucleoside may be selected from the antiviral nucleosides as disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety. In embodiments, the antiviral nucleosides is Compound A. In particular embodiments, the antiviral nucleoside is a mixture of Compound A and tautomers thereof. In other particular embodiments, the antiviral nucleoside is a mixture of Compound A, pharmaceutically acceptable salts of Compound A, tautomers of Compound A, and pharmaceutically acceptable salts of tautomers of Compound A.

An additional aspect of this embodiment relates to uses of a pharmaceutical composition in the preparation of a medicament for the treatment of viral infection, said pharmaceutical composition comprising (a) Compound A or a pharmaceutically acceptable salt thereof; and (b) a pharmaceutically acceptable carrier. In aspects of such embodiments, said pharmaceutical composition comprising (a) a mixture of Compound A and tautomers thereof, or pharmaceutically acceptable salts thereof; and (b) a pharmaceutically acceptable carrier.

The disclosure also provides uses of an antiviral compound in the preparation of a medicament for the treatment of viral infection, where the patient has previously (e.g., within 24 hours) been treated with another agent, which may be as described above.

In embodiments of the uses of an antiviral compound in the preparation of a medicament for the treatment of viral infection, the antiviral nucleoside is administered orally, by intravenous infusion, or by subcutaneous injection. In specific embodiments, the antiviral nucleoside is administered orally.

In embodiments of the uses of an antiviral compound in the preparation of a medicament for the treatment of viral infection, the antiviral nucleoside is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered once daily, as a single dose of an amount of about 200 mg. In aspects of such embodiments, the antiviral nucleoside is administered once daily, as a single dose of an amount of about 400 mg. In aspects of such embodiments, the antiviral nucleoside is administered once daily, as a single dose of an amount of about 600 mg. In aspects of such embodiments, the antiviral nucleoside once daily, as a single dose of administered at an amount of about 800 mg. In aspects of such embodiments, the antiviral nucleoside is administered once daily, as a single dose of an amount of about 1000 mg. In aspects of such embodiments, the antiviral nucleoside is administered once daily, as a single dose of an amount of about 1200 mg. In aspects of such embodiments, the antiviral nucleoside is administered once daily, as a single dose of an amount of about 1400 mg. In aspects of such embodiments, the antiviral nucleoside is administered once daily, as a single dose of an amount of about 1600 mg. In particular aspects of these embodiments, the antiviral nucleoside may be provided as individual doses using 1, 2, 3, 4, 5, 6, 7, or 8 200 mg capsules as individual unit dosage forms. In more specific embodiments, the antiviral nucleoside may be provided as individual doses using 4 200 mg capsules as individual unit dosage forms. In other specific embodiments, the antiviral nucleoside may be provided as individual doses using 8 200 mg capsules as individual unit dosage forms. In specific embodiments, the individual unit dosage forms are 200 mg capsules.

In embodiments of the uses of an antiviral compound in the preparation of a medicament for the treatment of viral infection, the antiviral nucleoside is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered twice daily, as individual doses of an amount of about 50 mg, about 100 mg, about 200 mg, about 300 mg, about 400 mg, about 500 mg, about 600 mg, about 700 mg, about 800 mg, or about 1000 mg. In aspects of such embodiments, the antiviral nucleoside is administered twice daily, as individual doses of an amount of about 200 mg. In aspects of such embodiments, the antiviral nucleoside is administered twice daily, as individual doses of an amount of about 400 mg. In aspects of such embodiments, the antiviral nucleoside is administered twice daily, as individual doses of an amount of about 600 mg. In aspects of such embodiments, the antiviral nucleoside is administered twice daily, as individual doses an amount of about 800 mg. In aspects of such embodiments, the antiviral nucleoside is administered twice daily, as individual doses of an amount of about 1000 mg. In particular aspects of these embodiments, the antiviral nucleoside may be provided as individual doses using 1, 2, 3, 4 or 5 200 mg capsules as individual unit dosage forms. In more specific embodiments, the antiviral nucleoside may be provided as individual doses using 4 200 mg capsules as individual unit dosage forms.

In embodiments of the uses of an antiviral compound in the preparation of a medicament for the treatment of viral infection, the antiviral nucleoside is administered once daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the antiviral nucleoside is administered once daily for 3 to 14 days, such as for 3 days, 4 days, 5 days, 6 days, 7 days, 8 days, 9 days, 10 days, 11 days, 12 days, 13 days, or 14 days. In specific embodiments, the antiviral nucleoside is administered once daily for 3 to 6 days, such as for 3 days, 4 days, 5 days, or 6 days. In another specific embodiment, the antiviral nucleoside is administered once daily for 5 days.

In embodiments of the uses of an antiviral compound in the preparation of a medicament for the treatment of viral infection, wherein the antiviral nucleoside is administered twice daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the antiviral nucleoside is administered twice daily for 3 to 14 days, such as for 3 days, 4 days, 5 days, 6 days, 7 days, 8 days, 9 days, 10 days, 11 days, 12 days, 13 days, or 14 days. In specific embodiments, the antiviral nucleoside is administered twice daily for 3 to 6 days, such as for 3 days, 4 days, 5 days, or 6 days. In another specific embodiment, the antiviral nucleoside is administered twice daily for 5 days.

The disclosure further relates to uses of an antiviral compound in the preparation of a medicament for the treatment of viral infection, wherein administration of the antiviral nucleoside is commenced 1 to 10 days after onset of symptoms of viral infection, such as 1 day, 2 days, 3 days, 4 days, 5 days, 6 days, 7 days, 8 days, 9 days, or 10 days after the onset of symptoms of viral infection. In specific embodiments, the antiviral nucleoside is administered beginning less than 5 days after onset of symptoms, such as less than 1 day, 2 days, 3 days, 4 days, or 5 days after onset of symptoms of viral infection. Symptoms of viral infection may be as described above.

The disclosure further relates to uses of an antiviral compound in the preparation of a medicament for the treatment of viral infection, wherein the antiviral nucleoside is administered to a subject who may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure further relates to uses of an antiviral compound in the preparation of a medicament for the treatment of viral infection, wherein administration of the antiviral nucleoside reduces the risk of hospitalization or death for the subject. In embodiments, administration of the antiviral nucleoside may result in a reduction in risk of hospitalization or death for the subject. In specific embodiments, administration of the antiviral nucleoside may result in a reduction in risk of hospitalization or death for the subject of about 1 to about 10 percent, such as from about 5 to about 7.5 percent, or about 6.8 percent. In further embodiments, administration of the antiviral nucleoside may result in a relative reduction in risk of hospitalization or death for the subject of up to about 50 percent.

The present disclosure also relates to uses of an antiviral compound in the preparation of a medicament for prevention of viral infection or providing antiviral prophylaxis, wherein the viral infection to be prevented is caused by a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In certain embodiments, the antiviral nucleoside may be selected from the antiviral nucleosides as disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety. In embodiments, the antiviral nucleosides is Compound A. In particular embodiments, the antiviral nucleoside is a mixture of Compound A and tautomers thereof. In other particular embodiments, the antiviral nucleoside is a mixture of Compound A, pharmaceutically acceptable salts of Compound A, tautomers of Compound A, and pharmaceutically acceptable salts of tautomers of Compound A.

An additional aspect of this embodiment relates to uses of a pharmaceutical composition in the preparation of a medicament for the prevention of viral infection or providing antiviral prophylaxis, said pharmaceutical composition comprising (a) Compound A or a pharmaceutically acceptable salt thereof; and (b) a pharmaceutically acceptable carrier. In aspects of such embodiments, said pharmaceutical composition comprising (a) a mixture of Compound A and tautomers thereof, or pharmaceutically acceptable salts thereof; and (b) a pharmaceutically acceptable carrier.

An additional aspect of this embodiment relates to uses of a pharmaceutical composition in the preparation of a medicament for the prevention of viral infection, said pharmaceutical composition comprising (a) Compound A or a pharmaceutically acceptable salt thereof; and (b) a pharmaceutically acceptable carrier. In aspects of such embodiments, said pharmaceutical composition comprising (a) a mixture of Compound A and tautomers thereof, or pharmaceutically acceptable salts thereof; and (b) a pharmaceutically acceptable carrier.

The disclosure also provides uses of an antiviral compound in the preparation of a medicament for the prevention of viral infection or providing antiviral prophylaxis, where the patient has previously (e.g., within 24 hours) been treated with another agent, which may be as described above.

In embodiments of the uses of an antiviral compound in the preparation of a medicament for the prevention of viral infection or providing antiviral prophylaxis, the antiviral nucleoside is administered orally, by intravenous infusion, or by subcutaneous injection. In specific embodiments, the antiviral nucleoside is administered orally.

The disclosure further relates to uses of an antiviral compound in the preparation of a medicament for the prevention of viral infection or antiviral prophylaxis, wherein the therapy commences prior to exposure to a viral infection or after a potential exposure to a viral infection. In specific embodiments, the antiviral nucleoside is administered beginning prior to potential exposure to viral infection. In other specific embodiments, the antiviral nucleoside is administered from 1 to days after potential exposure to a viral infection, such as 1 day, 2 days, 3 days, 4 days, 5 days, 6 days, 7 days, 8 days, 9 days, or 10 days after potential exposure. In specific embodiments, the antiviral nucleoside is administered beginning less than 5 days after potential exposure, such as less than 1 day, 2 days, 3 days, 4 days, or 5 days after potential exposure.

The disclosure further relates to uses of an antiviral compound in the preparation of a medicament for the prevention of viral infection or antiviral prophylaxis, wherein the antiviral nucleoside is administered to a subject who may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

Additionally, present disclosure relates to uses of an antiviral compound in the preparation of a medicament for the prevention of viral infection or antiviral prophylaxis, wherein the antiviral nucleoside is administered to a subject who may have been exposed to a virus selected from the group consisting of Eastern equine encephalitis virus, Western equine encephalitis virus, Venezuelan equine encephalitis virus, Chikungunya virus, Ross River virus, orthomyxoviridae virus, paramyxoviridae virus, RSV, influenza A virus, influenza B virus, filoviridae virus, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae virus, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of uses of an antiviral compound in the preparation of a medicament for the prevention of viral infection or antiviral prophylaxis, the antiviral nucleoside is administered orally, by intravenous infusion, or by subcutaneous injection. In specific embodiments, the antiviral nucleoside is administered orally.

In embodiments of uses of an antiviral compound in the preparation of a medicament for the prevention of viral infection or antiviral prophylaxis, the antiviral nucleoside is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered once daily, as a single dose of an amount of about 200 mg. In aspects of such embodiments, the antiviral nucleoside is administered once daily, as a single dose of an amount of about 300 mg. In aspects of such embodiments, the antiviral nucleoside is administered once daily, as a single dose of an amount of about 400 mg. In aspects of such embodiments, the antiviral nucleoside is administered once daily, as a single dose of an amount of about 500 mg. In aspects of such embodiments, the antiviral nucleoside is administered once daily, as a single dose of an amount of about 600 mg. In aspects of such embodiments, the antiviral nucleoside is administered once daily, as a single dose of an amount of about 700 mg. In aspects of such embodiments, the antiviral nucleoside once daily, as a single dose of administered at an amount of about 800 mg. In particular aspects of these embodiments, the antiviral nucleoside may be provided as individual doses using 1, 2, 3, or 4 200 mg capsules as individual unit dosage forms. In more specific embodiments, the antiviral nucleoside may be provided as individual doses using 4 200 mg capsules as individual unit dosage forms.

In embodiments of uses of an antiviral compound in the preparation of a medicament for the prevention of viral infection or antiviral prophylaxis, the antiviral nucleoside is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside is administered twice daily, as individual doses of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside is administered twice daily, as individual doses of an amount of about 200 mg. In aspects of such embodiments, the antiviral nucleoside is administered twice daily, as individual doses of an amount of about 300 mg. In aspects of such embodiments, the antiviral nucleoside is administered twice daily, as individual doses of an amount of about 400 mg. In aspects of such embodiments, the antiviral nucleoside is administered twice daily, as individual doses an amount of about 500 mg. In aspects of such embodiments, the antiviral nucleoside is administered twice daily, as individual doses of an amount of about 600 mg. In aspects of such embodiments, the antiviral nucleoside is administered twice daily, as individual doses of an amount of about 700 mg. In aspects of such embodiments, the antiviral nucleoside twice daily, as individual doses of administered at an amount of about 800 mg. In particular aspects of these embodiments, the antiviral nucleoside may be provided as individual doses using 1, 2, 3, or 4 200 mg capsules as individual unit dosage forms. In more specific embodiments, the antiviral nucleoside may be provided as individual doses using 4 200 mg capsules as individual unit dosage forms.

The disclosure further relates to uses of an antiviral compound in the preparation of a medicament for the prevention of viral infection or antiviral prophylaxis, wherein the antiviral nucleoside is administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside is administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside is administered once daily for 3 to 14 days, such as for 3 days, 4 days, 5 days, 6 days, 7 days, 8 days, 9 days, 10 days, 11 days, 12 days, 13 days, or 14 days.

The disclosure further relates to uses of an antiviral compound in the preparation of a medicament for the prevention of viral infection or antiviral prophylaxis, wherein the antiviral nucleoside is administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside is administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside is administered twice daily for 3 to 14 days, such as for 3 days, 4 days, 5 days, 6 days, 7 days, 8 days, 9 days, 10 days, 11 days, 12 days, 13 days, or 14 days.

Many patients with COVID-19 recover with no or minimal medical intervention. However, clinical progression to severe disease severely impacts both patients and healthcare systems, increasing an individual's risks of requiring mechanical ventilation and of death and potentially overburdening hospital capacity and available healthcare resources during COVID-19 surges. Reducing the number of patients requiring hospitalization for COVID-19 is therefore critical. Vaccination remains by far the most important medical intervention available to reduce the risk of hospitalization or death from COVID-19. However, early treatment soon after symptom onset has also been shown as effective. The monoclonal antibodies bamlanivimab/etesevimab, casirivimab/imdevimab, and sotrovimab are currently the only treatments authorized for at-risk outpatients with COVID-19. Because monoclonal antibodies require administration via infusion or injection in a medical setting, a direct-acting, oral agent such as Compound A that can be self-administered at home after diagnosis may be more practical for non-hospitalized patients and would be an important new tool in treating COVID-19 caused by SARS-CoV-2. Additional advantages of Compound A over monoclonal antibodies that attach to SARS-CoV-2 spike protein are Compound A's high barrier to development of resistance and its efficacy against SARS-CoV-2 variants; Compound A's proposed mechanism of action is independent of mutations in the spike protein, which categorize the various variants and can affect the efficacy of most monoclonal antibody treatments.

Additional embodiments of the disclosure include the pharmaceutical compositions, combinations, uses, and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### EXAMPLES

### Example 1: Randomized, Placebo-Controlled, Double-Blind Clinical Study to Evaluate the Efficacy, Safety, and Pharmacokinetics of Compound A in Hospitalized Adults with COVID-19

This was a randomized, placebo-controlled, double-blind, multisite study to evaluate the efficacy, safety, and PK of Compound A administered to hospitalized participants ≥18 years of age with PCR-confirmed COVID-19 and symptom onset within 10 days prior to randomization.

The trial was conducted in conformance with Good Clinical Practices.

Enrollment of participants with severe COVID-19 was limited to 50% of total planned sample size.

Participants received 10 doses of assigned study intervention by oral administration Q12H (±2 hours) and were followed for 28 days after the first dose (through Day 29).

### Dose Ranging

Using 200 mg capsules as the unit dose strength, 304 participants were randomized in a 1:1:1:1 ratio into 1 of the following 4 blinded treatment groups:
Compound A 200 mg (n=75)
Compound A 400 mg (n=75)
Compound A 800 mg (n=76)
Placebo (n=78)
   The final dose selection was based on analysis of data from this study in combination with the totality of data available across the Compound A clinical program.

### Throughout the Study

Participants received Sponsor-designated standard of care treatment of COVID-19, as appropriate, in addition to study intervention. All participants had plasma sample collection for PK assessments.

In the event of hospital discharge, study evaluations were performed. For participants who were discharged prior to end of treatment, a study intervention diary was used to record dose administration.

All participants have a virtual visit approximately 7 months following the last dose of study intervention (LFU visit) to assess survival status, current supplemental oxygen use, and to document any hospitalizations. In addition, pregnancy status for female participants of childbearing potential and female partners of male participants will be collected at this visit.

Interim analyses were reviewed by an independent eDMC and Sponsor siDMC. In addition, participant safety were monitored by the independent eDMC through periodic review of accumulating data (received from an unblinded statistician) as detailed in the eDMC charter.

### Scientific Rationale for Study Design

The primary outcome was assessed through Day 29 (for 28 days of follow-up from Day 1) to allow for a sufficient duration to assess the safety and effectiveness of a 5-day treatment course of Compound A.

### Primary Clinical Endpoint

The primary endpoint selected in this study, time-to-sustained recovery, was intended to demonstrate the efficacy of Compound A relative to placebo using a clinically meaningful aspect of the disease relevant to hospitalized patients with COVID-19. Sustained recovery is defined as:
Participant is alive and not hospitalized through Day 29 (including those re-hospitalized and discharged again before Day 29). This includes those discharged to home, home with nursing care, a rehabilitation facility, a long-term care facility, or a non-hospital intermediate care facility; or
Participant is alive and medically ready for discharge through Day 29 as determined by the investigator. This includes those hospitalized or rehospitalized participants who no longer require ongoing medical care but remain hospitalized for infection-control reasons or due to delay in identifying living accommodation outside the hospital.
   The following events did not meet the definition of sustained recovery:
Died, discharged to hospice care, remains in facility receiving ongoing medical care, transferred to a general hospital unit or another hospital for ongoing medical care, or re-hospitalized and not discharged or not medically ready for discharge again before Day 29.

### Secondary Endpoint (Mortality)

All-cause mortality was selected as a relevant secondary endpoint to further evaluate efficacy of Compound A when administered to participants hospitalized with COVID-19. All-cause mortality is of particular interest in this study population, given the significant mortality caused by COVID-19 and the need for therapeutic agents to demonstrate rapid attenuation early in disease progression.

### Secondary Endpoints (Ordinal Scales)

The Pulmonary and Pulmonary+ scales are ordinal categorical endpoints that assess intermediate measures of activity as indicators of disease progression and recovery. The Pulmonary ordinal scale focuses on the respiratory sequalae of COVID-19 and is defined based on oxygen requirements using 7 well-defined mutually exclusive categories. The Pulmonary+ ordinal scale is a 7-category assessment that captures the range of disease severity, including coagulation-related complications and respiratory dysfunction, experienced by hospitalized patients with COVID-19. The Pulmonary+ scale recognizes that non-pulmonary events are emerging as significant contributors to the overall morbidity of the disease. While the 2 scales are correlated, it was not pre-determined which of these 2 scales better represents clinical benefit and the impact of study intervention. Therefore, both scales were used to assess benefit.

In addition, the National Early Warning Score was used to assess a participant's degree of illness as assessed by clinical risk prediction categories for poor clinical outcomes including mortality within 24 hours of a set of vital sign measurements; the National Early Warning Score was used as supportive evidence of the efficacy of Compound A when administered in hospitalized participants with COVID-19.

To further evaluate efficacy of Compound A, the WHO 11-point ordinal outcome scale was also used to assess COVID-19-associated symptom burden (severity and duration), hospitalization, and death through Day 29.

### Safety Endpoints

Safety evaluations including AE collection, physical examinations (including vital signs) and laboratory tests (hematology and chemistry) were performed. AEs were evaluated and assessed according to the Division of AIDS (DAIDS) Table for Grading the Severity of Adult and Pediatric Adverse Events Corrected Version 2.1, July 2017 (downloaded from https://rsc.niaid.nih.gov/clinical-research-sites/grading-severity-adult-pediatric-adverse-events-corrected-version-two-one, on Sep. 11, 2020).

In preclinical studies, mild hematologic toxicity was noted on Day 7, which progressed to more severe pancytopenia after 14-21 days of continuous exposure at 2.2-fold and 0.5-fold the NHC exposure at the 200 mg Q12H and 800 mg Q12H human dose of Compound A respectively. These changes were fully reversible. There have been no clinically significant abnormalities observed in hematological laboratory tests in the Phase 1 study. However, based on pre-clinical findings, participants in this study was monitored for any signs of bone marrow toxicity, including monitoring of CBC and platelets after initiating study intervention.

In preclinical studies, elevated liver enzymes were noted in rats at 72-fold the NHC exposure at 800 mg Q12H and not noted in dogs at 22-fold the NHC exposure at 800 mg Q12H. No clinically significant abnormalities in liver parameters were noted in the Phase 1 study at any dose. However, elevated liver transaminases with a DILI pattern was considered an ECI and closely monitored.

No test-article related pathology changes in the pancreas to indicate pancreatic toxicity were noted in nonclinical studies, and lipase and amylase were not measured. Transient elevations of serum lipase were observed at least 3 days after last dose in Phase 1 study. The occurrence and magnitude of these elevations did not appear to be dose related, and they were not associated with abdominal/gastrointestinal symptoms. However, as asymptomatic lipase elevations and clinical pancreatitis have been associated with some nucleoside analogs, changes in amylase/lipase were considered an ECI and closely monitored.

### Pharmacokinetic Endpoints

Blood samples for PK assessment and concentrations of the Compound A nucleoside and triphosphate were collected from all participants. As appropriate, PK-efficacy and PK-AE relationships for Compound A were also evaluated. PBMC PK samples were used to evaluate the concentration of intracellular NHC-triphosphate, the active moiety resulting from dosing of Compound A. Intracellular PBMC concentrations can help explain the relationship between Compound A dose and efficacy and safety.

Plasma and PBMC PK parameters such as C_{trough}, Cₘₐₓ, tₘₐₓ, t_{1/2}, and AUC₀₋₁₂ were estimated.

### Virology Endpoints

The study evaluated SARS-CoV-2 RNA to assess the impact of Compound A on various aspects of SARS-CoV-2 viral dynamics.

Reducing SARS-CoV-2 viral load or eradicating the virus is essential to recovery and has important implications for transmission and infection control strategies. While not wanting to be bound by a single theory, as currently understood, the mechanism of antiviral activity of Compound A is viral error catastrophe predicated on increasing the viral mutation rate beyond a biologically-tolerable threshold resulting in impairment of viral fitness leading to viral extinction. These endpoints were aimed at assessing antiviral activity of Compound A as well as evaluating the rate of viral mutagenesis with Compound A treatment.

### Rationale for the Use of Comparator/Placebo

This study was placebo-controlled in order to avoid bias in the collection/evaluation of data during study conduct and to assess whether any observed effects are treatment-related or an impact of study participation. Participants may receive Sponsor-designated standard of care treatment as appropriate in addition to study intervention (Compound A or matching placebo).

There are currently no orally available direct-acting antivirals approved for the treatment of COVID-19.

### Rationale for the Selected Participant Population

The rationale for the participant population selected for this study was as follows:
Participants with mild, moderate, and severe COVID-19: Participants with mild, moderate, and severe COVID-19 may all require hospitalization and may benefit from administration of Compound A. Participants in this study must require medical care in the hospital for ongoing clinical manifestations of COVID-19 (not just for public health or quarantine purposes). Participants with critical COVID-19 will be excluded as antiviral therapy may have less impact on patients who have progressed to advanced critical illness due to the robust host inflammatory response that may confound antiviral treatment at this later stage in the disease. Furthermore, patients with critical COVID-19 demonstrated no difference in mortality or median time to recovery comparing placebo to remdesivir.
Participants with signs/symptoms attributable to COVID-19 for ≤10 days: Eligible participants must have COVID-19 signs/symptom onset no more than 10 days prior to randomization. SARS-CoV-2 viral loads are highest early in the course of disease, present 1-2 days prior to symptom onset, and persist for 7-12 days in moderate cases and up to 2 weeks in severe cases. Based on the viral kinetics of SARS-CoV-2 and the mechanism of action of Compound A (inhibition of viral replication), study participants must have signs/symptoms attributable to COVID-19 for ≤10 days prior to randomization with administration of the first dose occurring within 24 hours of randomization. Furthermore, as the host inflammatory response predominates during later stages of disease as COVID-19 progresses, treatment with antiviral therapy is likely to have a greater benefit with early treatment rather than delayed treatment >10 days after sign/symptoms onset.

### Justification for Dose—Rationale for Dosing Duration

Three doses of Compound A (administered every 12 hours) were sufficient to demonstrate efficacy in a ferret model of influenza. However, the duration of dosing of Compound A required to achieve efficacy against SARS-CoV-2 in humans was unknown. The planned treatment regimen of 5 days in this study was consistent with other acute antiviral treatments such as oseltamivir for influenza and was supported by nonclinical and clinical safety data for Compound A.

In a 28-day toxicity study of Compound A at 17 mg/kg/day (2.2-fold the predicted NHC exposure of 13.27 μM*hr at a dose of 200 mg BID) administered in dogs, reversible hematology changes consistent with bone marrow toxicity became apparent at Day 7 with increasing severity from Day 14 onward. Dosing of Compound A up to 800 mg Q12H for 5.5 days has been generally well tolerated by healthy participants in a Phase 1 clinical study; on review of preliminary blinded safety data, no clinically meaningful trends have been observed for changes in clinical laboratory values, vital signs, or ECGs as a function of dose or treatment. Specifically, there have been no clinically significant abnormalities observed in the hematological laboratory tests. Overall, preclinical and Phase 1 clinical observations to date support a ˜5-day dosing duration for Compound A.

### Dose Range

The dose range was derived based on the anticipated clinically efficacious dose range predicted from nonclinical animal models and from Phase 1 trials. Compound A demonstrated efficacy in ferrets (a relevant species for virus challenge models) against H1N1 at 7 mg/kg BID (in vitro data demonstrated similar Compound A potency against H1N1 and SARS-CoV-2). The efficacious 7 mg/kg BID dose in ferrets scales to ˜100 mg BID in humans, based on body surface area (assuming a 70 kg adult). This is a common scaling approach used for nucleosides, with some variability in the scaling of the prodrug to active triphosphate conversion from animals to humans.

The study includes evaluation of the 200 mg BID dose, as it is within the efficacious dose range predicted from animals, and it includes higher doses in order to characterize the dose- and exposure-response relationship for Compound A. The highest dose of 800 mg BID had a predicted steady-state mean plasma AUC₀₋₁₂ exposure of ˜32 μM*hr, which is 2.4-fold below the mean plasma AUC₀₋₁₂ exposure at the highest single dose evaluated in adults of 1600 mg.

Of 304 randomized participants (43.4% female, mean age 57.0 years), 218 received ≥1 dose of Compound A and 75 of placebo. Baseline characteristics were generally well balanced between study arms; 74.0% of participants had ≥1 risk factor for severe COVID-19. Drug-related adverse events were reported in 11.0% Compound A—treated and 21.3% placebo-treated participants, with no apparent dose effect on adverse event rates and no evidence of hematologic toxicity based on prespecified adverse events. Of 16 deaths, none were drug-related and most occurred in participants with severe COVID-19 (75.0%), with underlying comorbidities (87.5%), >60 years old (81.3%), and/or symptom duration >5 days (75.0%) at randomization. Median time to sustained recovery was 9 days in all arms, with similar day 29 recovery rates (81.5%-85.2%). At end of therapy, mean SARS-CoV-2 viral RNA mutation rates per 10,000 nucleotides were 5.9 with 800 mg Compound A versus 2.8 for placebo, consistent with Compound A's proposed mechanism of action. Compound A treatment resulted in more participants with undetectable SARS-CoV-2 RNA at day 10, especially among those with ≤5 days since sign/symptom onset (14.3%-36.4% more than placebo). In this phase 2 trial, a 5-day course of Compound A up to 800 mg twice daily was well tolerated and impacted relevant virologic measures, but clinical benefit was not observed in hospitalized patients with COVID-19.

Among participants with sequence data available, the SARS-CoV-2 genotype clades were determined to be those listed in Table 1.

TABLE 1

| | | | Compound | Compound | Compound |
|---|---|---|---|---|---|
| Clade | Total | Placebo | A, 200 | A, 400 | A, 800 |
| 20A, No. | 27 | 7 | 7 | 9 | 4 |
| 20B, No. | 64 | 16 | 14 | 15 | 19 |
| 20C, No. | 11 | 2 | 5 | 4 | 0 |
| 20D, No. | 1 | 0 | 1 | 0 | 0 |
| 20E, No. | 10 | 3 | 3 | 3 | 1 |
| 20G, No. | 6 | 0 | 1 | 3 | 2 |
| 20H (Beta), | 1 | 0 | 0 | 0 | 1 |
| No. | | | | | |
| 20I (Alpha), | 5 | 1 | 1 | 2 | 1 |
| No. | | | | | |
| Other or | 2 | 2 | 0 | 0 | 0 |
| Unknown | | | | | |

### Example 2: Randomized, Placebo-Controlled, Double-Blind Clinical Study to Evaluate the Safety, Tolerability, and Antiviral Efficacy of Compound a in Unvaccinated Participants with COVID-19

A phase 2a double-blind, placebo-controlled, randomized, multicenter clinical trial evaluated the safety, tolerability, and antiviral efficacy of Compound A in 202 unvaccinated participants with confirmed SARS-CoV-2 infection and symptom duration <7 days.

### Participants

Two hundred and two participants were randomized 1:1 to receive Compound A (200 mg) or placebo and then 3:1 to receive Compound A (400 or 800 mg) or placebo, orally twice daily for 5 days. Demographics and baseline characteristics are shown in Table 2 below.

TABLE 2

| | 200 mg | 400 mg | 800 mg | |
|---|---|---|---|---|
| | Compound A | Compound A | Compound A | Placebo |
| | N = 23 | N = 62 | N = 55 | N = 62 |
| Age, median (range), years | 32.0 (19-65) | 42.5 (19-82) | 42.0 (18-68) | 39.0 (19-71) |
| Age ≥65 years, n (%) | 1 (4.3) | 3 (4.8) | 4 (7.3) | 3 (4.8) |
| Sex, female, n (%) | 11 (47.8) | 32 (51.6) | 27 (49.1) | 34 (54.8) |
| Race, n (%) | | | | |
| Asian | 1 (4.3) | 2 (3.2) | 1 (1.8) | 2 (3.2) |
| Black or African American | 3 (13.0) | 3 (4.8) | 3 (5.5) | 2 (3.2) |
| White | 17 (73.9) | 56 (90.3) | 49 (89.1) | 54 (87.1) |
| Other | 2 (8.7) | 0 | 2 (3.6) | 1 (1.6) |
| Multiple | 0 | 1 (1.6) | 0 | 3 (4.8) |
| Ethnicity, Hispanic or Latino, n (%) | 7 (30.4) | 23 (37.1) | 33 (60.0) | 23 (37.1) |
| BMI, median, kg/m² | 25.50 | 26.70 | 27.00 | 27.05 |
| BMI ≥30 kg/m², n (%) | 7 (30.4) | 18 (29.0) | 15 (27.3) | 16 (25.8) |
| Baseline viral load, mean (SD), | 6.69 (1.888) | 6.38 (1.837) | 5.80 (1.823) | 6.11 (1.794) |
| log₁₀ copies/ml | | | | |
| Baseline viral load, median | 7.25 (3.0-9.5) | 6.72 (3.0-9.9) | 6.12 (3.0-9.4) | 6.40 (3.0-9.3) |
| (range), log₁₀ copies/ml | | | | |
| Days from symptom onset, | 4.00 (1.8-7.0) | 4.85 (2.5-7.1) | 4.60 (1.4-7.1) | 4.55 (1.8-7.5) |
| median (range), n | | | | |
| At least one risk factor for | 15 (65.2) | 37 (59.7) | 33 (60.0) | 37 (59.7) |
| severe disease, n (%) | | | | |
| SARS-CoV-2 Spike protein antibody | 3/20 (15.0) | 15/50 (30.0) | 18/51 (35.3) | 10/55 (18.2) |
| positive* on day 1, n/N† (%) | | | | |

BMI, body mass index; n, number of participants with response; N, number of participants in dose group; OD, optical density.
*SARS-CoV-2 Spike protein antibody positive includes testing positive for any of the following: total Ig, IgA, IgM, or IgG. A positive result was defined as an OD reading greater than 0.376 for total Ig, 0.3 for IgA, 0.31 for IgM, and 0.376 for IgG.
†N, number of participants with an antibody status

### Assessments

Antiviral activity was assessed by reverse transcriptase polymerase chain reaction (RT-PCR) for SARS-CoV-2 RNA in nasopharyngeal swabs. Infectious virus was assessed by inoculation of cultured Vero cells with samples from nasopharyngeal swabs and was detected by RT-PCR.

### Results

### Time to Clearance of SARS-CoV-2 Viral RNA in Nasopharyngeal Swabs

Time to clearance of viral RNA in nasopharyngeal swabs (<1018 copies/ml) was the primary endpoint of this study and was significantly reduced in participants receiving 800 mg of Compound A (median, 14 days) compared with those administered placebo (median, 15 days) (log-rank P value=0.013).

Time to clearance of viral RNA was not significantly different in participants who received 200 or 400 mg of Compound A compared to those who received placebo.

Given the difference in baseline antibody status between those who received Compound A 800 mg and placebo, sensitivity analysis evaluating only those participants who were seronegative at baseline was performed.

The reduction in time to clearance of viral RNA between 800 mg of Compound A and placebo in the seronegative participants was greater and remained significant (median, 14 days versus 27 days, respectively; P=0.001). The proportion of participants who achieved SARS-CoV-2 RNA clearance by day 28 (end of study) was also greater for those administered 800 mg of Compound A (92.5%) compared with those administered 200 mg of Compound A (91.3%), 400 mg of Compound A (78.7%), or placebo (80.3%) (Table 3).

TABLE 3
SARS-CoV-2 infectivity and virology.

| | | | |
|---|---|---|---|
| 200 mg | 400 mg | 800 mg | |
| Compound A | Compound A | Compound A | Placebo |

Percentage of participants positive for infectious SARS-CoV-2 virus

| Day 1, n/N (%) | 11/22 | (50.0) | 18/43 | (41.9) | 20/52 | (38.5) | 25/53 | (47.2) |
|---|---|---|---|---|---|---|---|---|
| Day 3, n/N (%) | 4/22 | (18.2) | 5/43 | (11.6) | 1/53 | (1.9) | 9/54 | (16.7) |
| Fisher's exact P value* | >0.99 | | 0.57 | | 0.016 | | | |
| Dose response P value | | | | | | | 0.010 | |
| Day 5, n/N (%) | 1/22 | (4.5) | 0/42 | (0.0) | 0/53 | (0.0) | 6/54 | (11.1) |

| Fisher's exact P value* | 0.67 | 0.034 | 0.027 | |
|---|---|---|---|---|
| Dose response P value | | | | 0.003 |

Time to SARS-CoV-2 viral RNA negativity

| Participants with response, | 21/23 | (91.3) | 48/61 | (78.7) | 49/53 | (92.5) | 49/61 | (80.3) |
|---|---|---|---|---|---|---|---|---|
| n/N (%) | | | | | | | | |
| Median time to response | 22.0 | (15.0-28.0) | 27.0 | (15.0-28.0) | 14.0 | (13.0-14.0) | 15.0 | (15.0-27.0) |

| (95% CI), days | | | |
|---|---|---|---|
| Log-rank P value* | 0.56 | 0.73 | 0.013 |

Change from baseline in SARS-CoV-2 viral load (log₁₀ copies/ml)

| Day 3, n/N | 23/23 | | 58/61 | | 51/53 | | 56/61 | |
|---|---|---|---|---|---|---|---|---|
| Least squares mean (SE) | −0.783 | (0.189) | −0.941 | (0.118) | −1.050 | (0.115) | −0.847 | (0.127) |
| Difference in least squares mean | 0.064 | | −0.094 | | −0.203 | | | |
| 95% CI | −0.397 to 0.525 | | −0.438 to 0.250 | | −0.543 to 0.137 | | | |
| P value | 0.78 | | 0.59 | | 0.24 | | | |
| Day 5, n/N | 23/23 | | 56/61 | | 52/53 | | 57/61 | |
| Least squares mean (SE) | −1.471 | (0.212) | −1.754 | (0.128) | −1.867 | (0.126) | −1.320 | (0.150) |
| Difference in least squares mean | −0.150 | | −0.434 | | −0.547 | | | |
| 95% CI | −0.674 to 0.374 | | −0.825 to −0.043 | | −0.935 to −0.159 | | | |
| P value | 0.57 | | 0.030 | | 0.006 | | | |
| Day 7, n/N | 23/23 | | 51/61 | | 49/53 | | 56/61 | |
| Least squares mean (SE) | −2.028 | (0.202) | −2.263 | (0.119) | −2.485 | (0.107) | −1.952 | (0.157) |
| Difference in least squares mean | −0.076 | | −0.311 | | −0.534 | | | |
| 95% CI | −0.591 to 0.438 | | −0.702 to 0.079 | | −0.910 to −0.157 | | | |
| P value | 0.77 | | 0.12 | | 0.006 | | | |
| Day 14, n/N | 23/23 | | 53/61 | | 48/53 | | 54/61 | |
| Least squares mean (SE) | −2.884 | (0.091) | −2.840 | (0.065) | −3.040 | (0.040) | −2.865 | (0.106) |

| Difference in least squares mean | −0.019 | 0.026 | −0.175 |
|---|---|---|---|
| 95% CI | −0.298 to 0.260 | −0.220 to 0.272 | −0.400 to 0.050 |
| P value | 0.89 | 0.84 | 0.13 |

*Comparison based on pooled placebo data.
n, number of observations; N, number of participants.

### Safety and Tolerability of Compound a

Compound A was associated with few, and mainly low-grade, adverse events that were similar to those reported by participants assigned to the placebo group. The incidence of treatment-associated adverse events was lowest in the 800-mg Compound A group. The only adverse events reported by more than four participants were headache, insomnia, and increased alanine

aminotransferase and there was no difference by treatment arm or dose. Two (1.4%) adverse events led to discontinuation of Compound A compared with one adverse event (1.6%) for placebo. Grade 3 or higher adverse events occurred in 5.0 and 8.1% of the combined Compound A groups and the placebo group, respectively. There were no dose-related trends in hematology or clinical chemistry data during the study.

Four serious adverse events occurred and resulted in hospitalization: One participant (1.6%) in the placebo group had hypoxia, two participants (3.2%) in the 400-mg Compound A group including one with a cerebrovascular accident and one with decreased oxygen saturation, and one participant (1.8%) in the 800-mg Compound A group had acute respiratory failure. Treatment was discontinued early in three of the four participants. The participant in the placebo group who experienced the serious adverse event of hypoxia died 31 days after onset of the serious adverse event.

Isolation of Infectious SARS-CoV-2 Virus from Nasopharyngeal Swabs

The secondary virological endpoint of this study was isolation of infectious virus from nasopharyngeal swabs and the reduction in time (days) to negativity. Infectious virus was isolated from 43.5% (74 of 170) of evaluable nasopharyngeal swabs at baseline (Table 3). On day 3 of treatment, infectious virus was isolated from only 1 of 53 (1.9%) participants administered 800 mg of Compound A compared with 9 of 54 (16.7%) participants administered placebo (P=0.016). At day 5 of treatment, infectious virus was not isolated from any participants receiving 400 or 800 mg of Compound A (0 of 42 and 0 of 53, respectively) compared with 11.1% of placebo recipients (6 of 54) of placebo recipients (P=0.034 and 0.027, respectively).

Change in SARS-CoV-2 Viral Load from Baseline

The decrease in viral RNA from baseline at study days 3, 5, 7, and 14 was greater for the 800-mg Compound A group than for any of the other groups at each time point (Table 3). For participants administered 400 or 800 mg of Compound A, the least squares mean viral load change from baseline was significantly greater on day 5 when compared with the placebo group, with differences in least squares means of −0.434 and −0.547 log₁₀ copies/ml (P=0.030 and 0.006), respectively. In addition, for participants administered 800 mg of Compound A, the least squares mean viral load change from baseline was also significantly greater on day 7 compared with the placebo group, with a least squares mean difference of −0.534 log₁₀ copies/ml (P=0.006).

### Example 3: Randomized, Placebo-Controlled, Double-Blind Clinical Study to Evaluate the Efficacy, Safety, and Pharmacokinetics of Compound a in Non-Hospitalized Adults with COVID-19

This was a randomized, placebo-controlled, double-blind, multi-site study to evaluate the efficacy, safety and PK of Compound A administered to non-hospitalized participants ≥18 years of age with laboratory-confirmed COVID-19 and symptom onset within 7 days (Part 1) or 5 days (Part 2) prior to randomization.

In Part 1 (Phase 2), enrolled participants had mild, laboratory-confirmed COVID-19, and had symptom onset within 7 days prior to randomization, and at least 75% of participants overall had at least 1 characteristic or underlying medical condition associated with being at increased risk for severe illness from COVID-19. In addition, enrollment of participants with moderate COVID-19 were limited to approximately 50% of total planned sample size.

In Part 2 (Phase 3), all participants had laboratory-confirmed COVID-19, and had symptom onset within 5 days prior to randomization, and at least 1 characteristic or underlying medical condition associated with being at increased risk for severe illness from COVID-19. Age is a risk factor independent of other health conditions, so while there was no minimal enrollment for participants >60 years of age, every effort was made to maximize enrollment of this age group.

Participants received assigned study intervention by oral administration Q12H for 5 days, for a total of 10 doses and be followed for 28 days after randomization (through Day 29). In addition, participants will be contacted approximately 7 months after the last dose of study intervention.

The trial was conducted in conformance with Good Clinical Practices.

### Part 1 (Dose Ranging)

Using 200 mg capsules as the unit dose strength, 302 participants were randomized in a 1:1:1:1 ratio into 1 of the following 4 blinded treatment groups:
Compound A 200 mg (n=75)
Compound A 400 mg (n=77)
Compound A 800 mg (n=76)
Placebo (n=74)

The final dose selection was based on analysis of data from this study in combination with the totality of data available across the Compound A clinical program prior to initiating Part 2. The study enrolled a total of 1433 participants as it was stopped due to early efficacy.

### Part 2 (Evaluation of Selected Dose)

In Part 2, the study anticipated a total of 1550 participants. Participants were randomized in a 1:1 ratio to receive either the selected dose of Compound A or placebo. Interim efficacy analysis was planned in Part 2, and the primary efficacy analysis was met at IA4, where 775 participants were randomized.

### Throughout the Study (Parts 1 and 2)

Participants received Sponsor-designated standard of care treatment of COVID-19, as appropriate, in addition to study intervention. Plasma samples were collected from all participants for PK assessments.

Participants were provided with a Study Medication Diary (dose administration were to be recorded daily for 5 days) and a Symptom Diary (presence/absence and severity of a solicited list of signs/symptoms attributable to COVID-19 were to be recorded daily for 29 days). In the event of hospitalization, all evaluations were performed as feasible.

All participants will have a virtual visit approximately 7 months following the last dose of study intervention (LFU visit) to assess survival status, current supplemental oxygen use, and to document any hospitalizations. In addition, pregnancy status for female participants of childbearing potential and female partners of male participants will be collected at this visit.

Interim analyses were reviewed by an independent eDMC and Sponsor siDMC. In addition, participant safety was monitored by the independent eDMC through periodic review of accumulating data (received from an unblinded statistician) as detailed in the eDMC charter.

### Scientific Rationale for Study Design

The randomized, placebo-controlled, double-blind superiority design and the selected endpoints of the study were consistent with regulatory guidance and were considered appropriate for hospitalized adult participants with COVID-19.

The primary outcome was assessed through Day 29 (for 28 days of follow-up from Day 1) to allow for a sufficient duration to reliably assess the safety and effectiveness of a 5-day treatment course of Compound A.

### Primary Clinical Endpoint

The primary endpoint selected in this study was hospitalization or death. All-cause hospitalization (≥24 hours of acute care in a hospital or similar acute care facility, including emergency rooms or facilities created to address hospitalization needs during the COVID-19 pandemic) or death was intended to demonstrate the efficacy of Compound A relative to placebo using a clinically meaningful aspect of the disease that is relevant to non-hospitalized patients with COVID-19. This endpoint combined key clinical outcomes of interest, aiming to demonstrate the efficacy of study intervention in reducing serious complications of COVID-19 disease.

### Secondary Endpoints

To further evaluate efficacy of Compound A administered to non-hospitalized participants, additional relevant endpoints were selected based on signs/symptoms associated with COVID-19 infection and other aspects of clinical progression of disease that are expected to improve with effective antiviral therapy:
Time to improvement/resolution, and time to progression of targeted self-reported signs/symptoms attributable to COVID-19 as reported by the participant through Day 29.
WHO 11-point ordinal outcome scale to assess COVID-19-associated symptom burden (severity and duration), hospitalization, and death through Day 29.

### Safety Endpoints

Safety evaluations included AE collection, physical examinations (including vital signs) and laboratory tests (hematology and chemistry). AEs were evaluated and assessed according to the Division of AIDS (DAIDS) Table for Grading the Severity of Adult and Pediatric Adverse Events Corrected Version 2.1, July 2017 (downloaded from https://rsc.niaid.nih.gov/clinical-research-sites/grading-severity-adult-pediatric-adverse-events-corrected-version-two-one, on Sep. 11, 2020).

In preclinical studies, mild hematologic toxicity was noted on Day 7, which progressed to more severe pancytopenia after 14-21 days of continuous exposure at 2.2-fold and 0.5-fold the NHC exposure at the 200 mg Q12H and 800 mg Q12H human dose of Compound A respectively. These changes were fully reversible. There were no clinically significant abnormalities observed in hematological laboratory tests in the Phase 1 study. However, based on pre-clinical findings, participants in this study were monitored for any signs of bone marrow toxicity, including monitoring of CBC and platelets after initiating study intervention. There were no clinically significant abnormalities observed in hematological laboratory tests in Part 1 (Phase 2) or Part 2 (Phase 3).

In preclinical studies, elevated liver enzymes were noted in rats at 72-fold the NHC exposure at 800 mg Q12H and not noted in dogs at 22-fold the NHC exposure at 800 mg Q12H. No clinically significant abnormalities in liver parameters were noted in the Phase 1 study at any dose. However, elevated liver transaminases with a DILI pattern were considered an ECI and closely monitored. No clinically significant abnormalities in liver parameters were noted in the Part 1 (Phase 2) or Part 2 (Phase 3) at any dose.

No test-article related pathology changes in the pancreas to indicate pancreatic toxicity were noted in nonclinical studies and lipase and amylase were not measured. Transient elevations of serum lipase were observed at least 3 days after last dose in the Phase 1 study; the incidence of these elevations was comparable between recipients of Compound A and placebo. The occurrence and magnitude of these elevations did not appear to be dose related, and they were not associated with abdominal/gastrointestinal symptoms. However, as asymptomatic lipase elevations and clinical pancreatitis have been associated with some nucleoside analogs, changes in amylase or lipase were considered an ECI and closely monitored in Part 1 of this study. Based on available unblinded data from the clinical development program for Compound A, no clinically significant abnormalities in amylase or lipase as a function of either dose or treatment were noted. Thus due to the lack of preclinical findings and no trend noted in Compound A studies in Part 1, elevated amylase, or lipase >3× the upper limit of normal is not considered an ECI for Part 2.

### Pharmacokinetic Endpoints

Blood samples for PK assessment and concentrations of the Compound A nucleoside and triphosphate were collected from all participants. As appropriate, PK-efficacy and PK-AE relationships for Compound A will also be evaluated. In Part 1, PBMC PK parameters (e.g., C_{trough}) were estimated. In Part 2, plasma PK parameters (e.g., C_{trough}) were summarized, and in Parts 1 and 2, PK plasma parameters were measured. PBMC PK samples were used to evaluate the concentration of intracellular NHC-triphosphate, the active moiety resulting from dosing of Compound A. Intracellular PBMC concentrations can help explain the relationship between Compound A dose and efficacy and safety.

### Virology Endpoints

The study evaluated SARS-CoV-2 RNA to assess the impact of Compound A on various aspects of SARS-CoV-2 viral dynamics.

Reducing SARS-CoV-2 viral load or eradicating the virus is essential to recovery and has important implications for transmission and infection control strategies. While not wanting to be bound by a single theory, as currently understood, the mechanism of antiviral activity of Compound A is viral error catastrophe predicated on increasing the viral mutation rate beyond a biologically-tolerable threshold resulting in impairment of viral fitness leading to viral extinction. These endpoints were aimed at assessing antiviral activity of Compound A as well as evaluating the rate of viral mutagenesis with Compound A treatment.

### Rationale for the Use of Comparator/Placebo

This study was placebo-controlled in order to avoid bias in the collection/evaluation of data during study conduct and to assess whether any observed effects are treatment-related or an impact of study participation. Participants received Sponsor-designated standard of care treatment as appropriate in addition to study intervention (Compound A or matching placebo).

### Rationale for the Selected Participant Population

The rationale for the participant population selected for this study was as follows:
Participants with mild to moderate COVID-19: Participants with mild and moderate COVID-19 are typically managed outside of the hospital and may benefit from administration of Compound A. Participants with severe and critical COVID-19 at randomization were excluded because they were expected to require hospitalization and would be out of scope for this study.
Participants at increased risk for severe illness from COVID-19: Certain characteristics or underlying medical conditions have been identified that place patients at increased risk for severe illness from COVID-19, which may result in hospitalization, ICU-level care, ventilatory support, or death. This study focused on higher risk non-hospitalized participants who may benefit from Compound A, aiming to reduce the number of participants who develop severe illness from COVID-19 and either die or need to be hospitalized. Thus, in Part 1 (Phase 2), enrolled participants had mild, laboratory-confirmed COVID-19, and had symptom onset within 7 days prior to randomization, and at least 75% of participants overall had at least 1 characteristic or underlying medical condition associated with being at increased risk for severe illness from COVID-19 to ensure the drug was being evaluated in the non-hospitalized population at higher risk. Similarly, in Part 2, all participants had laboratory-confirmed COVID-19, and had symptom onset within 5 days prior to randomization, and at least 1 characteristic or underlying medical condition associated with being at increased risk for severe illness from COVID-19.
Participants with signs/symptoms attributable to COVID-19 for ≤5 days: Eligible participants must have had COVID-19 signs/symptoms onset no more than 5 days prior to randomization. SARS-CoV-2 viral loads are highest early in the course of disease, present 1-2 days prior to symptom onset, and persist for 7-12 days in moderate cases and up to 2 weeks in severe cases. Based on the viral kinetics of SARS-CoV-2 and the mechanism of action of Compound A (inhibition of viral replication), study participants must have had signs/symptoms attributable to COVID-19 for ≤5 days prior to randomization with administration of the first dose occurring within 24 hours of randomization.

### Justification for Dose-Rationale for Dosing Duration

Three doses of Compound A (administered every 12 hours) were sufficient to demonstrate efficacy in a ferret model of influenza. However, the duration of dosing of Compound A required to achieve efficacy against SARS-CoV-2 in humans was previously unknown. The planned treatment regimen of 5 days in this study was consistent with other acute antiviral treatments such as oseltamivir for influenza and was supported by nonclinical and clinical safety data for Compound A.

In a 28-day toxicity study of Compound A at 17 mg/kg/day (2.2-fold the predicted NHC exposure of 13.27 μM*hr at a dose of 200 mg BID) administered in dogs, reversible hematology changes consistent with bone marrow toxicity became apparent at Day 7 with increasing severity from Day 14 onward. Dosing of Compound A up to 800 mg Q12H for 5.5 days has been generally well tolerated by healthy participants in a Phase 1 clinical study; on review of preliminary blinded safety data, no clinically meaningful trends have been observed for changes in clinical laboratory values, vital signs, or ECGs as a function of dose or treatment. Specifically, there have been no clinically significant abnormalities observed in the hematological laboratory tests. Furthermore, unblinded data obtained from participants treated in the Phase 2 program indicate that Compound A has been generally well-tolerated across all doses studied. Overall, preclinical and Phase 1 clinical observations supported a ˜5-day dosing duration for Compound A.

### Dose Range for Part 1 (Phase 2)

The dose range planned for Part 1 was derived based on the anticipated clinically efficacious dose range predicted from nonclinical animal models and from Phase 1 trials. Compound A demonstrated efficacy in ferrets (a relevant species for virus challenge models) against H1N1 at 7 mg/kg BID (in vitro data demonstrated similar Compound A potency against H1N1 and SARS-CoV-2). The efficacious 7 mg/kg BID dose in ferrets scales to ˜100 mg BID in humans, based on body surface area (assuming a 70 kg adult). This is a common scaling approach used for nucleosides, with some variability in the scaling of the prodrug to active triphosphate conversion from animals to humans.

Part 1 of the study included evaluation of the 200 mg BID dose, as it was within the efficacious dose range predicted from animals, and it included higher doses in order to characterize the dose- and exposure-response relationship for Compound A. The highest dose of 800 mg BID had a predicted steady-state mean plasma AUC₀₋₁₂ exposure of ˜32 μM*hr, which is 2.4-fold below the mean plasma AUC₀₋₁₂ exposure at the highest single dose evaluated in adults of 1600 mg.

### Part 1 Analysis

Enrollment of Part 1 (Phase 2) has been completed. A total of 302 participants were randomized into 4 intervention groups: 75 participants in the Compound A 200 mg group, 77 participants in the Compound A 400 mg group, 76 participants in the Compound A 800 mg group, and 74 participants in the placebo group. Compound A was well-tolerated in non-hospitalized participants in Part 1. The proportion of participants with AEs, drug-related AEs, SAEs, AEs leading to death, and AEs leading to study intervention discontinuation during the protocol-specified AE safety follow-up period were comparable across the intervention groups, with no apparent dose effect observed. No ECIs were reported and there were no clinically meaningful abnormalities in hematological, pancreatic, or hepatic parameters as a function of either dose or treatment.

Among participants with sequence data available, the SARS-CoV-2 genotype clades were determined to be those listed in Table 4.

TABLE 4

| | | | Compound | Compound | Compound |
|---|---|---|---|---|---|
| Clade | Total | Placebo | A, 200 | A, 400 | A, 800 |
| 20A, No. | 36 | 9 | 6 | 12 | 9 |
| 20B, No. | 63 | 15 | 18 | 11 | 19 |
| 20C, No. | 20 | 6 | 6 | 2 | 6 |
| 20D, No. | 1 | 0 | 0 | 1 | 0 |
| 20E, No. | 8 | 2 | 3 | 2 | 1 |
| 20G, No. | 23 | 5 | 7 | 8 | 3 |
| 20H (Beta), | 4 | 2 | 0 | 1 | 1 |
| No. | | | | | |
| 20I (Alpha), | 1 | 1 | 0 | 0 | 0 |
| No. | | | | | |
| Other or | 0 | 0 | 0 | 0 | 0 |
| Unknown | | | | | |

The eDMC review of unblinded IA2 data from Part 1 concluded that there were no safety signals seen at any dose, and no dose-limiting toxicity was observed at the highest dose (800 mg). Furthermore, Compound A has been generally well-tolerated in other studies in the Compound A program at all doses studied with no dose-limiting toxicity observed at the highest dose (800 mg).

Virology data from IA2 in the Compound A program show that treatment with Compound A reduces the SARS-CoV-2 VL compared with placebo (based on change from baseline, slope of decline, and greater proportion of participants with a VL below the limit of quantitation within 15 or 29 days) in nonhospitalized participants enrolled in Part 1 and participants with symptom onset ≤7 days in Part 1. The exposure-response analysis for various virologic endpoints based on Part 1 suggests that the 800 mg Q12H dose provides a larger magnitude of virologic effect compared to 200 and 400 mg Q12H and is near the plateau of the dose-response curve. In addition, consistent with the proposed mechanism of action of Compound A of viral error catastrophe, the highest percentage of mutations in viral RNA post-treatment at Day 5 were observed in the 800 mg Q12H intervention group from Part 1.

Evaluation of the primary clinical efficacy endpoint for Part 1 showed that 11 of 299 participants were hospitalized through Day 29 (including 1 participant treated with placebo who died); ˜ 3% of participants in the Compound A intervention groups were hospitalized or died through Day 29 (compared to ˜5% in the placebo group). All hospitalized participants had at least 1 risk factor for severe illness from COVID-19 including but not limited to obesity (n=8), >60 years of age (n=5), and diabetes (n=5). Protocol-specified subgroup analyses for the primary endpoint indicated potential clinical benefit from treatment with Compound A early in the course of disease (i.e., symptom onset ≤7 days prior to the day of randomization) as well as in individuals with risk factors for severe illness from COVID-19, including age >60 years. This trend is further supported by exposure-response analyses for the endpoint of hospitalization which suggest a trend of an increased clinical effect at Compound A 800 mg Q12H dose over placebo or lower Compound A doses.

### Dose Selection for Part 2

The Compound A dose of 800 mg Q12H for 5 days was selected for Part 2 of this study. The dose selection was based on the totality of the observed safety profile and virologic data in the Compound A program, and trends in clinical efficacy in Part 1, the 800 mg Q12H dose was selected as the dose for further evaluation in Part 2 (Phase 3).

### Trial Design and Randomization

Part 2 was a randomized, placebo-controlled, double-blind phase 3 trial initiated on May 7, 2021 (first participant screened), evaluating the safety and efficacy of Compound A in non-hospitalized adults with COVID-19. The trial was conducted at 166 hospitals/treatment centers in 23 countries, following a previously completed phase 2 component.

Eligible participants were randomized 1:1, via a centralized interactive response technology system, to placebo or Compound A 800 mg (four 200 mg capsules), to be administered twice daily for 5 days. Randomization was stratified (block size: 4) by TSSO (≤3 days, >3 days). Participants, investigators, and study staff will remain blinded to treatment assignment until study completion.

### Oversight

The trial was conducted in accordance with principles of Good Clinical Practice and was approved by the appropriate institutional review boards/ethics committees and regulatory agencies. Written informed consent was obtained from all participants. Safety oversight was performed by an independent data monitoring committee.

### Assessments

COVID-19 signs/symptoms (i.e., cough, sore throat, nasal congestion, runny nose, shortness of breath or difficulty breathing, muscle or body aches, fatigue/tiredness, feeling hot/feverish, chills, headache, nausea, vomiting, diarrhea, loss of taste, loss of smell) were self-reported daily by participants from randomization through Day 29 via a paper diary. Nasopharyngeal swabs (for quantitation of SARS-CoV-2 RNA via polymerase chain reaction (PCR) and baseline sequencing for viral genotyping) were collected on Days 1, 3, 5, 10, 15, and 29; hospitalization status, vital signs, laboratory tests, and physical examinations were also assessed on those days. Adverse events (AEs) were assessed during treatment and through 14 days following end of treatment; serious AEs considered drug-related by the investigator were collected through the end of study participation.

### Outcomes

The primary efficacy endpoint was the percentage of participants with all-cause hospitalization (i.e., ≥24 hours of acute care in a hospital or any similar facility) and/or death through Day 29, in the modified intent-to-treat (MITT) population (i.e., all randomized participants who received ≥1 dose of study intervention and were not hospitalized before the first dose). The co-primary safety endpoint was evaluation of safety, i.e., the percentage of participants with AEs in the safety population (i.e., all randomized participants who received ≥1 dose of study treatment). Safety events of clinical interest were any post-baseline platelet values <50,000/μL and potential drug-induced liver injury (defined as either aspartate aminotransferase or alanine aminotransferase ≥3 times the upper limit of normal (ULN), plus total bilirubin ≥2 times the ULN, plus alkaline phosphatase <2 times the ULN).

Secondary efficacy endpoints included time to sustained resolution or improvement (number of days from randomization to the first of 3 consecutive days of resolution/improvement, without subsequent relapse by Day 29) and time to progression number of days from randomization to the first of 2 consecutive days of worsening) of each COVID-19 sign/symptom (reported as not present, mild, moderate, or severe). Improvement and progression were defined as any reduction or worsening, respectively, of baseline symptom severity. Exploratory endpoints included mean change in SARS-CoV-2 viral load from baseline.

### Statistical Analysis

Formal evaluation (via hypothesis testing) of the efficacy of Compound A relative to placebo was based on the difference (Compound A minus placebo) in the percentages of participants meeting the primary efficacy endpoint, assessed by the stratified (by TSSO: ≤3 days vs >3 days) Miettinen and Nurminen method. Missing mortality status at Day 29 was counted as having an outcome of hospitalization or death. Time to hospitalization/death, as well as time to sustained symptom resolution/improvement and progression, were compared between intervention groups using the stratified log-rank test and a Cox proportional hazard model. All analyses were conducted using SAS version 9.4 (SAS Institute, Cary, NC). Differences in SARS-CoV-2 RNA titer change from baseline over time were estimated using longitudinal models.

The planned enrollment of 1550 participants provided >95% power to demonstrate superiority in the primary endpoint at a one-sided 2.5% alpha level if the underlying event rates were 6% and 12% in the Compound A and placebo groups, respectively. An interim analysis based on the primary efficacy endpoint, with the main purpose of evaluating for early efficacy or futility, was pre-specified to occur once 50% of participants had been followed through Day 29. Strong control of the type I error rate at an overall one-sided 0.025 level was achieved by using efficacy boundaries determined through the Gamma family spending function, with γ=−1 (corresponding to a p-value boundary for efficacy of 0.0092 based on the number of participants in the efficacy analysis at the time of the interim analysis). No additional hypothesis testing was planned for other endpoints. In alignment with the pre-specified statistical analysis plan, 95% confidence intervals (CIs) were calculated for other select endpoints (not adjusted for multiple comparisons).

### Part 2 IA3/IA4 Results

### Participants

This interim analysis (database lock: Sep. 18, 2021) included 775 randomized participants: 387 randomized to Compound A and 388 to placebo. Baseline demographics and clinical characteristics were generally comparable between intervention groups as illustrated in Table 5. Overall, 49.2% had TSSO≤3 days, 43.4% had moderate COVID-19, and the most common risk factors were obesity (76.5%), age >60 years (13.7%), and diabetes mellitus (13.5%). Positive SARS-CoV-2 antibody results (as measured by the Elecsys® Anti-SARS-CoV-2-nucleocapsid assay (Roche Diagnostics, Indianapolis, IN, USA)) at baseline, indicating recent/prior infection, were reported for 18.2%. Among participants with sequence data available at the time of database lock (277/775; 35.7%), the 3 most common SARS-CoV-2 genotype clades were Mu (35.0%), Delta (22.4%), and Gamma (22.4%). Almost all randomized participants (98.3%; 385 Compound A, 377 placebo) were included in the MITT population; 10 (1 Compound A, 9 placebo) did not receive study intervention and 3 (1 Compound A, 2 placebo) were hospitalized prior to first dose. In those who received study intervention, average treatment duration was 4.4 days in both groups and most (95.3% Compound A, 93.4% placebo) received ≥9 doses.

TABLE 5
Baseline demographics and clinical characteristics

| | | | |
|---|---|---|---|
| | Compound A | Placebo | Total |
| Participants in population | N = 387 | N = 388 | N = 775 |

Sex

| | | | | | | |
|---|---|---|---|---|---|---|
| Female, No. (%) | 200 | (51.7) | 171 | (44.1) | 371 | (47.9) |

Age (years)

| | | | | | | |
|---|---|---|---|---|---|---|
| ≤60, No. (%) | 336 | (86.8) | 333 | (85.8) | 669 | (86.3) |
| >60, No. (%) | 51 | (13.2) | 55 | (14.2) | 106 | (13.7) |

Race

| | | | | | | |
|---|---|---|---|---|---|---|
| American Indian/Alaska Native, | 20 | (5.2) | 9 | (2.3) | 29 | (3.7) |
| No. (%) | | | | | | |
| Asian, No. (%) | 7 | (1.8) | 11 | (2.8) | 18 | (2.3) |
| Black or African American, No. | 27 | (7.0) | 20 | (5.2) | 47 | (6.1) |
| (%) | | | | | | |
| White, No. (%) | 194 | (50.1) | 209 | (53.9) | 403 | (52.0) |
| Multiple, No. (%) | 139 | (35.9) | 139 | (35.8) | 278 | (35.9) |

Ethnicity

| | | | | | | |
|---|---|---|---|---|---|---|
| Hispanic or Latino, No. (%) | 224 | (57.9) | 228 | (58.8) | 452 | (58.3) |
| Not Hispanic or Latino, No. (%) | 163 | (42.1) | 159 | (41.0) | 322 | (41.5) |

Region

| | | | | | | |
|---|---|---|---|---|---|---|
| North America, No. (%) | 15 | (3.9) | 22 | (5.7) | 37 | (4.8) |
| Latin America, No. (%) | 216 | (55.8) | 214 | (55.2) | 430 | (55.5) |
| Europe, No. (%) | 89 | (23.0) | 90 | (23.2) | 179 | (23.1) |
| Asia Pacific, No. (%) | 5 | (1.3) | 6 | (1.5) | 11 | (1.4) |
| Africa, No. (%) | 62 | (16.0) | 56 | (14.4) | 118 | (15.2) |

Risk factors for severe illness from COVID-19

| | | | | | | |
|---|---|---|---|---|---|---|
| At least one risk factor, No. (%) | 385 | (99.5) | 384 | (99.0) | 769 | (99.2) |
| Obesity (BMI ≥30), No. (%) | 306 | (79.1) | 287 | (74.0) | 593 | (76.5) |
| Age >60 years, No. (%) | 51 | (13.2) | 55 | (14.2) | 106 | (13.7) |
| Diabetes mellitus, No. (%) | 48 | (12.4) | 57 | (14.7) | 105 | (13.5) |
| Serious heart condition, No. (%) | 42 | (10.9) | 36 | (9.3) | 78 | (10.1) |
| Chronic kidney disease, No. (%) | 14 | (3.6) | 20 | (5.2) | 34 | (4.4) |
| COPD, No. (%) | 7 | (1.8) | 22 | (5.7) | 29 | (3.7) |
| Active cancer, No. (%) | 6 | (1.6) | 11 | (2.8) | 17 | (2.2) |

Baseline Disease Severity

| | | | | | | |
|---|---|---|---|---|---|---|
| Mild, No. (%) | 222 | (57.4) | 212 | (54.6) | 434 | (56.0) |
| Moderate, No. (%) | 162 | (41.9) | 174 | (44.8) | 336 | (43.4) |
| Severe or Unknown,^{c} No. (%) | 3 | (0.8) | 2 | (0.5) | 5 | (0.6) |

Baseline clade designation (variant)

| | | | | | | |
|---|---|---|---|---|---|---|
| 19B, No. (%) | 1 | (0.3) | 1 | (0.3) | 2 | (0.3) |
| 20A, No. (%) | 3 | (0.8) | 2 | (0.5) | 5 | (0.6) |
| 20B, No. (%) | 4 | (1.0) | 4 | (1.0) | 8 | (1.0) |
| 20D, No. (%) | 2 | (0.5) | 1 | (0.3) | 3 | (0.4) |
| 20H (Beta), No. (%) | 5 | (1.3) | 6 | (1.5) | 11 | (1.4) |
| 20I (Alpha), No. (%) | 12 | (3.1) | 8 | (2.1) | 20 | (2.6) |
| 20J (Gamma), No. (%) | 35 | (9.0) | 48 | (12.4) | 83 | (10.7) |
| 21A (Delta), No. (%) | 96 | (24.8) | 90 | (23.2) | 186 | (24.0) |
| 21G (Lambda), No. (%) | 12 | (3.1) | 7 | (1.8) | 19 | (2.5) |
| 21H (Mu), No. (%) | 70 | (18.1) | 80 | (20.6) | 150 | (19.4) |
| 21I (Delta), No. (%) | 6 | (1.6) | 3 | (0.8) | 9 | (1.2) |
| 21J (Delta), No. (%) | 12 | (3.1) | 15 | (3.9) | 27 | (3.5) |
| Other,^{a} No. (%) | 2 | (0.5) | 2 | (0.5) | 4 | (0.5) |
| Sequence not yet available, No. (%) | 127 | (32.8) | 121 | (31.2) | 248 | (32.0) |

Time from COVID-19 sign/symptom onset to randomization^{b}

| | | | | | | |
|---|---|---|---|---|---|---|
| ≤3 days, No. (%) | 191 | (49.4) | 190 | (49.0) | 381 | (49.2) |

SARS-CoV-2 RNA at baseline in nasopharyngeal sample (qualitative assay)

| | | | | | | |
|---|---|---|---|---|---|---|
| Detectable, No. (%) | 332 | (85.8) | 331 | (85.3) | 663 | (85.5) |
| Undetectable, No. (%) | 28 | (7.2) | 29 | (7.5) | 57 | (7.4) |
| Unknown^{c}, No. (%) | 27 | (7.0) | 28 | (7.2) | 55 | (7.1) |

SARS-CoV-2 baseline antibody

| | | | | | | |
|---|---|---|---|---|---|---|
| Positive, No. (%) | 71 | (18.3) | 70 | (18.0) | 141 | (18.2) |
| Negative, No. (%) | 299 | (77.3) | 288 | (74.2) | 587 | (75.7) |
| Unknownc, No. (%) | 17 | (4.4) | 30 | (7.7) | 47 | (6.1) |

^{a}Includes the following clades: 20C and clade unknown/could not be classified.
^{b}Based on data collected to stratify at randomization.
^{c}Missing data, invalid sample, tests not done, or results reported as “unknown” are categorized as unknown.

### Efficacy

At the interim analysis, Compound A met the criteria for demonstration of superiority; the percentage of participants in the MITT population who were hospitalized or died through Day 29 was statistically significantly lower in the Compound A (7.3%) than in the placebo group (14.1%). Treatment with Compound A resulted in a 6.8 percentage point reduction (95% CI: −11.3, −2.4; p=0.001) in the risk of hospitalization or death through Day 29 compared with placebo. Time-to-event analyses were consistent with these primary analysis results: Compound A treatment resulted in a ˜50% reduction in the risk of hospitalization/death through Day 29 with a hazard ratio of 0.51 (95% CI: 0.32, 0.81). The benefit of treatment with Compound A appeared to begin around Day 3 and increased thereafter, with the largest differences in the cumulative incidence rate of hospitalization or death observed at Day 10 through Day 29. No participants in the Compound A group died through Day 29; all 8 participants who died through Day 29 were in the placebo group (a Day 29 mortality rate of 2.1%) and were hospitalized prior to their death. Of note, only 5 of all hospitalizations/deaths were not considered COVID related by the investigators; outcomes in a post-hoc sensitivity analysis including only MITT participants with COVID-19-related hospitalizations/deaths were fully consistent with the primary analysis (Table 6). Subgroup analyses (in subgroups that were predefined based on baseline characteristics) were consistent with the overall primary endpoint analysis, except in the subgroup of participants positive for SARS-CoV-2 antibodies at baseline (indicative of a recent or prior SARS-CoV-2 infection and occurring at comparable rates in both groups) where there was no difference between intervention groups for the primary endpoint (2.9% in both groups). Due to these compelling efficacy (exceeding the pre-specified efficacy boundary) and safety results, the independent data monitoring committee of the trial recommended an early stop to trial recruitment.

TABLE 6
Post-hoc sensitivity analysis of the primary endpoint, comparing
rates of specifically COVID-19-related hospitalizations
and/or deaths through Day 29 (MITT population)

| | | | Compound A vs. placebo | |
|---|---|---|---|---|
| | | | Unadjusted | Adjusted difference in |
| Treatment | No. | n (%) | difference | rates % (95% CI)^{a} |
| Compound A | 385 | 25 (6.5) | −6.8 | −6.8 (−11.1, −2.6) |
| Placebo | 377 | 50 (13.3) | | |

^{a}Adjusted differences and the corresponding confidence intervals are based on Miettinen & Nurminen method stratified by randomization strata.
MITT, modified intent-to-treat. n, number of participants who died or were hospitalized through Day 29. No., number of participants in the MITT population.
Unknown survival status at Day 29 was not counted as having an outcome of COVID-related hospitalization or death.

A higher percentage of participants in the Compound A group achieved earlier sustained improvement/resolution for most of the self-reported COVID-19 signs/symptoms. Similarly, a lower percentage of participants in the Compound A than the placebo group reported progression of most COVID-19 signs/symptoms. Changes in the WHO Clinical Progression Scale also suggested clinical benefit with Compound A treatment, with a greater percentage of placebo group participants than Compound A group participants exhibiting worse outcomes at Days 5, 10, and 15.

### Virology

Testing for evaluation of virologic response was ongoing at the time of the interim analysis. Compound A treatment was associated with significantly greater reductions in viral load from baseline at Days 3 and 5 than placebo; results at other timepoints were generally comparable between groups (Table 7). Results by baseline SARS-CoV-2 RNA titer (>10⁶ and ≤10⁶ copies/mL) were generally consistent with the overall results for the mean change from baseline in SARS-CoV-2 RNA, with participants who had >10⁶ copies/mL showing the greatest reductions (Table 7).

TABLE 7
Mean change from baseline over time in
SARS-CoV-2 RNA titer (MITT population)

| | Compound A | | | Placebo | | |
|---|---|---|---|---|---|---|
| | | | Mean | | | Mean |
| | | | change^{a} | | | change^{a} |
| Visit | No. | Mean^{a} | (SD) | No. | Mean^{a} | (SD) |

SARS-CoV-2 RNA Titer (log¹⁰ copies/ml)

| | | | | | | | | |
|---|---|---|---|---|---|---|---|---|
| Baseline | 314 | 6.85 | | | 304 | 6.99 | | |
| Day 3 | 301 | 5.82 | −1.04 | (1.263) | 290 | 6.16 | −0.82 | (1.211) |
| EOT (Day 5) | 285 | 4.80 | −2.09 | (1.471) | 286 | 5.29 | −1.72 | (1.485) |
| Day 10 | 267 | 3.70 | −3.18 | (1.660) | 263 | 3.86 | −3.11 | (1.645) |
| Day 15 | 266 | 3.20 | −3.69 | (1.754) | 245 | 3.32 | −3.61 | (1.755) |
| Day 29 | 186 | 2.90 | −4.08 | (1.644) | 193 | 2.91 | −4.22 | (1.635) |

SARS-CoV-2 RNA titer (log¹⁰ copies/ml) in participants with
baseline RNA titer ≤10⁶ copies/ml

| | | | | | | | | |
|---|---|---|---|---|---|---|---|---|
| Baseline | 86 | 4.58 | | | 76 | 4.60 | | |
| Day 3 | 83 | 3.88 | −0.67 | (1.255) | 74 | 4.43 | −0.16 | (1.292) |
| EOT (Day 5) | 76 | 3.56 | −1.00 | (1.175) | 71 | 3.67 | −0.89 | (1.481) |
| Day 10 | 71 | 3.05 | −1.49 | (1.018) | 68 | 3.16 | −1.37 | (1.222) |
| Day 15 | 70 | 3.01 | −1.51 | (1.159) | 67 | 3.07 | −1.54 | (1.228) |
| Day 29 | 47 | 2.81 | −1.76 | (1.070) | 43 | 2.85 | −1.72 | (1.003) |

SARS-CoV-2 RNA titer (log¹⁰ copies/ml) in participants with
baseline RNA titer >10⁶ copies/ml

| | | | | | | | | |
|---|---|---|---|---|---|---|---|---|
| Baseline | 228 | 7.71 | | | 228 | 7.79 | | |
| Day 3 | 218 | 6.56 | −1.17 | (1.242) | 216 | 6.75 | −1.04 | (1.098) |
| EOT (Day 5) | 209 | 5.25 | −2.49 | (1.367) | 215 | 5.82 | −2.00 | (1.384) |
| Day 10 | 196 | 3.94 | −3.79 | (1.406) | 195 | 4.10 | −3.71 | (1.312) |
| Day 15 | 196 | 3.26 | −4.47 | (1.180) | 178 | 3.41 | −4.39 | (1.203) |
| Day 29 | 139 | 2.93 | −4.87 | (0.886) | 150 | 2.93 | −4.93 | (0.923) |

^{a}Mean and mean change from baseline is based on the measurements of participants with values at both baseline and the time point assessed.
No., Number of participants with baseline and at least one postbaseline test result at the time point assessed. SD, Standard deviation.
Analysis only includes participants with baseline SARS-CoV-2 RNA titer ≥500 copies/mL. The quantitative assay to generate these data was the Q2 SARS-CoV-2 Viral Load Quantitation Assay, with lower limit of quantification of 500 copies/ml and upper limit of quantification of 500,000,000 copies/ml. Postbaseline results above or below these limits were included in the mean and mean change from baseline, with imputed value 499 and 500,000,001, respectively. Day 3 includes post-baseline records up to Day 4. EOT (Day 5) includes post-baseline records from Day 5 up to Day 7. EOT visits occurring carlier than Day 5 are included in the Day 3 visit.

### Safety

The percentage of participants with ≥1 AE was comparable between the Compound A (35.0%) and placebo (39.6%) groups, as was the percentage of participants with drug-related AEs (12.4% vs 11.1%, respectively). Serious AEs, none of which were deemed drug-related by the 5 investigator, and serious AEs leading to discontinuation of study intervention were less frequently reported among participants treated with Compound A (Table 8). No deaths were reported with Compound A in the interim analysis, while 8 deaths occurred in the placebo arm through Day 29. Two additional deaths as a result of reported AEs occurred in the placebo arm after Day 29. Results for the safety endpoints of serious AEs and deaths, which overlap with the primary efficacy 10 endpoint, were consistent with the primary efficacy analysis results.

TABLE 8
Incidence of adverse event rates (safety population)

| | | | | | Estimated | |
|---|---|---|---|---|---|---|
| | | | | | difference vs | |
| | Compound A | | Placebo | | placebo, % | |
| | n | (%) | n | (%) | (95% CI)^{a} | |
| Participants in safety population | 386 | | 379 | | | |
| At least 1 AE | 135 | (35.0) | 150 | (39.6) | −4.6 | (−11.4, 2.3) |
| At least 1 drug-related^{b} AE | 48 | (12.4) | 42 | (11.1) | 1.4 | (−3.3, 6.0) |
| At least 1 serious AE | 28 | (7.3) | 53 | (14.0) | −6.7 | (−11.2, −2.4) |
| At least 1 serious drug-related AE | 0 | (0.0) | 0 | (0.0) | 0.0 | (−1.0, 1.0) |
| Died | 0 | (0.0) | 10 | (2.6) | −2.6 | (−4.8, −1.4) |
| Discontinued treatment due to an AE | 5 | (1.3) | 13 | (3.4) | −2.1 | (−4.6, 0.0) |
| Discontinued treatment due to a | 3 | (0.8) | 3 | (0.8) | −0.0 | (−1.6, 1.6) |
| drug-related AE | | | | | | |
| Discontinued treatment due to a | 1 | (0.3) | 9 | (2.4) | −2.1 | (−4.2, −0.6) |
| serious AE | | | | | | |
| Discontinued treatment due to a | 0 | (0.0) | 0 | (0.0) | 0.0 | (−1.0, 1.0) |

serious drug-related AE

AE, adverse event. n, number of participants per intervention arm and row title.
^{a}Based on the Miettinen & Nurminen method.
^{b}Determined by the investigator to be related to study treatment.

The most frequently reported AEs (≥2% participants in either group; Compound A versus placebo, respectively) were worsening COVID-19 (8.0% versus 14.8%), COVID-19 pneumonia (4.9% versus 9.0%), diarrhea (3.9% versus 4.5%), nausea (2.8% versus 1.3%), bacterial pneumonia (2.1% versus 0.8%), and respiratory failure (1.0% versus 2.1%). The most frequently reported drug-related (as per investigator assessment) AEs were diarrhea (3.1% versus 3.2%) and nausea (2.3% versus 1.1%). No participants in the Compound A group met prespecified criteria for platelets <50,000/μL or potential drug-induced liver injury.

### Analysis of Part 2

The interim analysis of Part 2, randomized, controlled phase 3 trial in non-hospitalized at-risk adults with COVID-19 demonstrated that Compound A is superior to placebo in reducing the risk of all-cause hospitalization and/or mortality through Day 29. The study population was representative of real-world patients with one or more well-established risk factors for severe illness due to COVID-19. There was a nearly 50% relative reduction in the percentage of participants who were hospitalized or died through Day 29, a substantial improvement in an outcome that is meaningful to patients, healthcare systems, and public health. The efficacy benefit with Compound A treatment was consistent across important patient subgroups, including participants infected with the SARS-CoV-2 variants of concern Delta, Gamma, and Mu. Notably, there were no deaths with Compound A, but 3% of placebo-treated participants died. Patient-reported data suggested improvements in COVID-19 symptoms with Compound A compared to placebo. Compound A was well-tolerated; no safety concerns were identified, and there was no evidence of clinically meaningful abnormalities in laboratory tests. Due to these compelling efficacy and safety results, the independent data monitoring committee of the trial recommended an early stop to trial recruitment.

In summary, Compound A is a safe and effective, orally administered treatment for COVID-19 in non-hospitalized adults at risk of progression to severe disease, significantly reducing their mortality and need for hospitalization.

### Final Results

### Eligible Participants

Nonhospitalized adults with mild or moderate Covid-19 were eligible; mild or moderate illness was determined on the basis of definitions adapted from Food and Drug Administration (COVID-19: developing drugs and biological products for treatment or prevention: guidance for industry. Silver Spring, MD: Food and Drug Administration, May 2020 (https://www.fda.gov/regulatory-information/search-fda-guidance-documents/covid-19-developing-drugs-and-biological-products-treatment-or-prevention) and World Health Organization (WHO) guidance (WHO COVID-19 case definitions. Geneva: World Health Organization, Dec. 16, 2020 (https://apps.who.int/iris/rest/bitstreams/1322790/retrieve).

Key inclusion criteria at randomization were SARS-CoV-2 infection that had been laboratory-confirmed no more than 5 days earlier, onset of signs or symptoms no more than 5 days earlier, at least one sign or symptom of Covid-19, and at least one risk factor for development of severe illness from Covid-19 (age >60 years; active cancer; chronic kidney disease; chronic obstructive pulmonary disease; obesity, defined by a body-mass index [the weight in kilograms divided by the square of the height in meters]≥30; serious heart conditions [heart failure, coronary artery disease, or cardiomyopathies]; or diabetes mellitus).

Key exclusion criteria were an anticipated need for hospitalization for Covid-19 within the next 48 hours, dialysis or estimated glomerular filtration rate less than 30 ml per minute per 1.73 m2, pregnancy, unwillingness to use contraception during the intervention period and for at least 4 days after completion of the regimen, severe neutropenia (absolute neutrophil count of <500 per milliliter), platelet count below 100,000 per microliter, and SARS-CoV-2 vaccination.

Standard-of-care treatment with antipyretic agents, antiinflammatory agents, glucocorticoids, or a combination was permitted; use of therapies intended as Covid-19 treatments (including any monoclonal antibodies and remdesivir) was prohibited through day 29.

### Participants

The final, all-randomized analysis sample included a total of 1433 participants who were enrolled at 107 sites in 20 countries and underwent randomization. With the exception of sex, baseline demographic and clinical characteristics were generally similar in the two groups at the time of both the interim analysis (Table 5) and the final all-randomized analysis (Table 9). More women were randomly assigned to Compound A, and the imbalance was larger in the interim analysis sample (difference, 7.6 percentage points) than in the all-randomized sample (difference, 4.7 percentage points). The participants were largely representative of the expected patient population. Overall, 47.7% of the participants had had onset of signs or symptoms 3 days or less before randomization and 44.5% had moderate Covid-19. The most common risk factors were obesity (73.7%), age over 60 years (17.2%), and diabetes mellitus (15.9%). SARS-CoV-2 nucleocapsid antibodies at baseline, indicating recent or previous infection (not vaccination), were reported among 19.8% of participants. Owing to the ongoing nature of testing, 25.9% of the participants in the interim analysis sample did not have baseline sequence data available, and 44.7% in the all-randomized sample did not have baseline sequence data available at the time of this report. Among all participants who underwent randomization and had sequence data available, the three most common SARS-CoV-2 variants were B.1.617.2 (delta; 58.1%), B.1.621 (mu; 20.5%), and P.1 (gamma; 10.7%). Almost all the participants (98.3%; 709 in the Compound A group and 699 in the placebo group) were included in the modified intention-to-treat population. Among those who received Compound A or placebo, most (95.2% in the Compound A group and 94.7% in the placebo group) received at least 9 doses.

Survival status at day 29 was confirmed for all but a single participant in the modified intention-to-treat population, including participants who discontinued the trial early; this single participant (in the placebo group), whose hospitalization status at day 29 was also unknown, was the only one for whom the primary end point was imputed as hospitalized or dead. For two additional participants, hospitalization status at day 29 could not be determined; these participants were confirmed to be alive at day 29 and were therefore counted as alive and not hospitalized through day 29, in accordance with the prespecified analysis plan.

TABLE 9
Baseline demographics and clinical characteristics *

| | | Compound A | Placebo | Total |
|---|---|---|---|---|
| Participants in population | | N = 716 | N = 717 | N = 1433 |
| Female sex - no. (%) | | 384 (53.6) | 351 (49.0) | 735 (51.3) |
| Age group - no. (%) | | | | |
| 18-49 | yr | 484 (67.6) | 465 (64.9) | 949 (66.2) |
| ≥50 | yr | 232 (32.4) | 252 (35.1) | 484 (33.8) |
| Median age (range) - yr | | 42.0 (18-90) | 44.0 (18-88) | 43.0 (18-90) |
| Risk factors for severe illness | | | | |
| from Covid-19 - no. (%) | | | | |
| At least one risk factor | | 712 (99.4) | 712 (99.3) | 1424 (99.4) |
| Obesity† | | 538 (75.1) | 518 (72.2) | 1056 (73.7) |
| Age >60 | yr | 119 (16.6) | 127 (17.7) | 246 (17.2) |

| | | | |
|---|---|---|---|
| Diabetes mellitus | 107 (14.9) | 121 (16.9) | 228 (15.9) |
| Serious heart condition | 86 (12.0) | 81 (11.3) | 167 (11.7) |
| Chronic kidney disease | 38 (5.3) | 46 (6.4) | 84 (5.9) |
| Chronic obstructive pulmonary disease | 22 (3.1) | 35 (4.9) | 57 (4.0) |
| Active cancer | 13 (1.8) | 16 (2.2) | 29 (2.0) |
| Covid-19 severity - no. (%) | | | |
| Mild | 395 (55.2) | 390 (54.4) | 785 (54.8) |
| Moderate | 315 (44.0) | 323 (45.0) | 638 (44.5) |
| Severe or unknown‡ | 6 (0.8) | 4 (0.6) | 10 (0.7) |
| Clade designation; variant - no. (%) | | | |
| 20H; beta | 5 (0.7) | 6 (0.8) | 11 (0.8) |
| 20I; alpha | 12 (1.7) | 9 (1.3) | 21 (1.5) |
| 20J; gamma | 37 (5.2) | 48 (6.7) | 85 (5.9) |
| 21A, 21I, 21J; delta | 237 (33.1) | 223 (31.1) | 460 (32.1) |
| 21G; lambda | 14 (2.0) | 7 (1.0) | 21 (1.5) |
| 21H; mu | 76 (10.6) | 86 (12.0) | 162 (11.3) |
| Other§ | 16 (2.2) | 16 (2.2) | 32 (2.2) |
| Evaluable sequence data not yet available | 319 (44.6) | 322 (44.9) | 641 (44.7) |
| Time from onset of Covid-19 signs or symptoms to | 342 (47.8) | 342 (47.7) | 684 (47.7) |
| randomization of ≤3 days - no. (%)¶ | | | |
| SARS-CoV-2 RNA in nasopharyngeal sample, | | | |
| qualitative assay - no. (%)‡ | | | |
| Detectable | 615 (85.9) | 615 (85.8) | 1230 (85.8) |
| Undetectable | 54 (7.5) | 51 (7.1) | 105 (7.3) |
| SARS-CoV-2 nucleocapsid antibody - no. (%)‡∥ | | | |
| Positive | 137 (19.1) | 147 (20.5) | 284 (19.8) |
| Negative | 541 (75.6) | 521 (72.7) | 1062(74.1) |

* Participants are those who underwent randomization.
†Obesity was defined by a body-mass index of 30 or higher.
‡Missing data, invalid samples, tests not done, or results reported as “unknown” are all categorized as unknown and are not shown individually.
§“Other” includes the following clades: 19B, 20A, 20B, 20C, 20D, and unknown clades or those that could not be classified.
¶ The time period was based on data collected at randomization.
∥Data are based on nucleocapsid antibody assay and do not reflect prior vaccination status, since Covid-19 vaccines generate antibodies against the SARS-CoV-2 spike protein, not the SARS-CoV-2 nucleocapsid protein.

### Efficacy

At day 29, the percentage of participants in the modified intention-to-treat population who had been hospitalized or had died was significantly lower in the Compound A group (7.3% [28 of 385 participants]) than in the placebo group (14.1% [53 of 377 participants), a treatment difference of −6.8 percentage points (95% confidence interval [CI], −11.3 to −2.4; P=0.001). In the all-randomized modified intention-to-treat population, participants receiving Compound A had a lower risk of hospitalization or death through day 29:6.8% (48 of 709 participants) in the Compound A group as compared with 9.7% (68 of 699 participants) in the placebo group (difference, −3.0 percentage points; 95% CI, −5.9 to −0.1).

A prespecified supporting analysis specifically evaluating only Covid-19-related hospitalizations or deaths showed that 45 of 709 participants (6.3%) in the Compound A group and 64 of 699 (9.2%) in the placebo group had hospitalizations or deaths that were considered by the investigators to be Covid-19-related (difference, −2.8 percentage points; 95% CI, −5.7 to 0.0). The results of a post hoc analysis adjusted for participant sex (the only baseline factor potentially unbalanced between the groups) were consistent with those of the primary analysis, with a risk of hospitalization or death through day 29 that was lower by 2.8 percentage points (95% CI, −5.7 to 0.1) with Compound A over placebo.

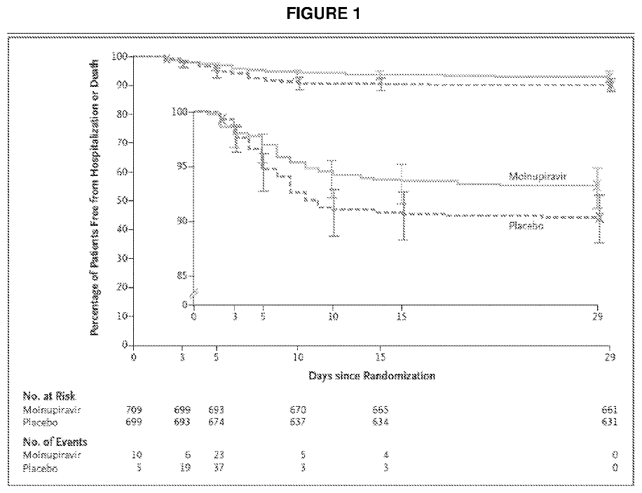

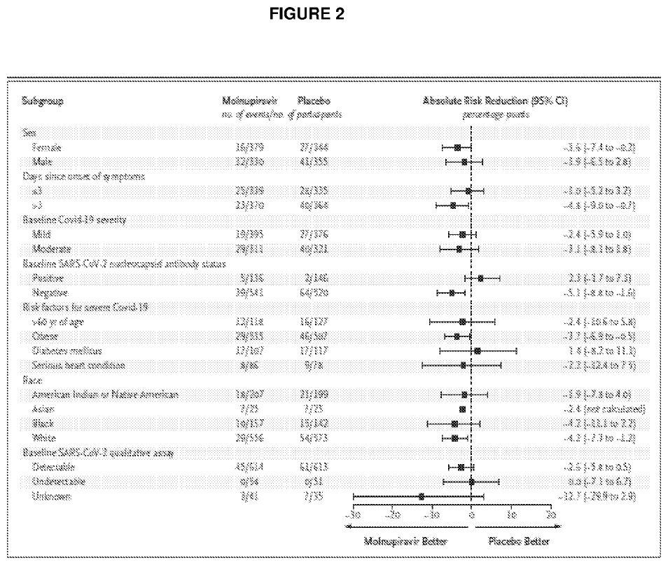

The results of a time-to-event analysis were also consistent with the primary results; the rate of hospitalization or death through day 29 was approximately 31% lower with Compound A than with placebo (hazard ratio, 0.69; 95% CI, 0.48 to 1.01) (FIG. 1). One death was reported in the Compound A group (29-day all-cause mortality, 0.1%) and 9 deaths in the placebo group (29-day all-cause mortality, 1.3%), a risk of death that was lower by 89% (95% CI, 14 to 99) with Compound A than with placebo. All 10 participants had been hospitalized before death, and all the deaths were considered by the investigators to be Covid-19-related. In most prespecified subgroups, the percentage of participants who were hospitalized or died was lower with Compound A than placebo, but the associated confidence intervals indicate substantial uncertainty about the magnitude of these effects (FIG. 2). The point estimate for the difference in the risk of hospitalization or death through day 29 favored placebo over Compound A only in patients with SARS-CoV-2 nucleocapsid antibodies at baseline; patients with low viral load at baseline; patients with diabetes at baseline; patients who identified themselves as Asian only, Black only, Native American only, or mixed Black-Native American-White; and patients enrolled in the Asia-Pacific region; all the associated 95% confidence intervals included zero and some were fairly wide, particularly because some of these subgroups had small sample sizes.

On the basis of the WHO Clinical Progression Scale, a greater percentage of participants in the Compound A group than in the placebo group showed improved outcomes by day 5, with the largest differences observed by days 10 and 15 (Table 10). For most Covid-19 signs and symptoms, sustained abatement or resolution was more likely and progression of signs or symptoms was less likely in the Compound A group than in the placebo group.

The WHO Clinical Progression Scale is an 11-point ordinal scale (ranging from 0 through 10), measuring the clinical progression of Covid-19.6 The minimally important difference is not known. Scores are assigned as follows:
Score 0: Uninfected, no SARS-CoV-2 viral RNA detected.
Score 1: Asymptomatic, but SARS-CoV-2 viral RNA detected.
Score 2: Symptomatic, but able to function independently.
Score 3: Symptomatic and assistance needed.
Score 4: Hospitalized, without oxygen therapy. [Note: Patients hospitalized for observation only were assigned a score of 1, 2, or 3, depending on their symptoms or condition, as per those respective scoring categories.]
Score 5: Hospitalized and administered oxygen by mask or nasal prongs.
Score 6: Hospitalized and administered oxygen by non-invasive ventilation or high flow.
Score 7: Intubated and receiving mechanical ventilation, PiO₂/FiO₂≥150 or SpO₂/FiO₂≥200.
Score 8: Receiving mechanical ventilation PiO₂/FiO₂<150 (SpO₂/FiO₂<200) or vasopressors.
Score 9: Receiving mechanical ventilation PiO₂/FiO₂<150 and vasopressors, dialysus, or extracorporeal membrane oxygenation.
Score 10: Dead.

In Table 10, odds ratios were estimated using the proportional odds model with WHO 11-point Clinical Progression Score categories as the response variable. Day 3 includes post-baseline records up to day 4 relative to randomization. Day 5 includes post-baseline records from day 5 (relative to randomization) up to day 7. End of treatment visits occurring earlier than day 5 (relative to randomization) are included in the day 3 visit.

| | | Compound A | | Placebo | |
|---|---|---|---|---|---|
| | Score | N = 709 | | N = 699 | |
| Visit | Category | no./m (%) | | no./m (%) | |
| Baseline | 0 | 0/706 | (0.0) | 0/695 | (0.0) |
| | 1-3 | 706/706 | (100.0) | 695/695 | (100.0) |
| | 4-5 | 0/706 | (0.0) | 0/695 | (0.0) |
| | 6-9 | 0/706 | (0.0) | 0/695 | (0.0) |
| | 10 | 0/706 | (0.0) | 0/695 | (0.0) |
| | Missing | 3 | | 4 | |
| Day 3 | 0 | 2/695 | (0.3) | 3/684 | (0.4) |
| | 1-3 | 679/695 | (97.7) | 663/684 | (96.9) |
| | 4-5 | 11/695 | (1.6) | 17/684 | (2.5) |
| | 6-9 | 3/695 | (0.4) | 1/684 | (0.1) |
| | 10 | 0/695 | (0.0) | 0/684 | (0.0) |
| | Missing | 14 | | 15 | |
| | Odds | 1.19 (0.62, 2.30) | | | |
| | ratio | | | | |
| | (95% CI) | | | | |
| Day 5 | 0 | 11/697 | (1.6) | 10/684 | (1.5) |
| | 1-3 | 663/697 | (95.1) | 636/684 | (93.0) |
| | 4-5 | 17/697 | (2.4) | 34/684 | (5.0) |
| | 6-9 | 6/697 | (0.9) | 4/684 | (0.6) |
| | 10 | 0/697 | (0.0) | 0/684 | (0.0) |
| | Missing | 12 | | 15 | |
| | Odds | 1.52 (0.96, 2.39) | | | |
| | ratio | | | | |
| | (95% CI) | | | | |
| Day 10 | 0 | 40/673 | (5.9) | 32/673 | (4.8) |
| | 1-3 | 599/673 | (89.0) | 580/673 | (86.2) |
| | 4-5 | 27/673 | (4.0) | 44/673 | (6.5) |
| | 6-9 | 7/673 | (1.0) | 17/673 | (2.5) |
| | 10 | 0/673 | (0.0) | 0/673 | (0.0) |
| | Missing | 36 | | 26 | |
| | Odds | 1.58 (1.14, 2.20) | | | |
| | ratio | | | | |
| | (95% CI) | | | | |
| Day 15 | 0 | 102/669 | (15.2) | 94/667 | (14.1) |
| | 1-3 | 548/669 | (81.9) | 525/667 | (78.7) |
| | 4-5 | 15/669 | (2.2) | 33/667 | (4.9) |
| | 6-9 | 4/669 | (0.6) | 10/667 | (1.5) |
| | 10 | 0/669 | (0.0) | 5/667 | (0.7) |
| | Missing | 40 | | 32 | |
| | Odds | 1.36 (1.03, 1.78) | | | |
| | ratio | | | | |
| | (95% CI) | | | | |
| Day 29 | 0 | 312/645 | (48.4) | 314/650 | (48.3) |
| | 1-3 | 324/645 | (50.2) | 314/650 | (48.3) |
| | 4-5 | 6/645 | (0.9) | 12/650 | (1.8) |
| | 6-9 | 2/645 | (0.3) | 1/650 | (0.2) |
| | 10 | 1/645 | (0.2) | 9/650 | (1.4) |

| Missing | 64 | 49 |
|---|---|---|
| Odds | 1.04 (0.84, 1.29) | |
| ratio | | |
| (95% CI) | | |

CI, confidence interval.
m, number of participants with non-missing ordinal scale at the time point assessed.
no., number of participants in each subcategory.
N, number of participants in each treatment group.

### Virology

At the time of the all-randomized analysis, 1093 of 1408 participants (77.6%) in the modified intention-to-treat population had quantifiable RNA confirmed in nasopharyngeal samples at baseline and, of those, samples from 964 participants (88.2%) had been tested at day 5. On the basis of the available data, Compound A treatment was associated with greater reductions from baseline in mean viral load than placebo at days 3, 5 (end-of-treatment visit), and 10. Results at other time points were similar in the two groups.

### Safety

The percentage of participants with at least one adverse event was similar in the two groups (30.40% in the Compound A group and 33.0% in the placebo group), as was the percentage of participants with adverse events considered by the investigators to be related to the trial regimen (8.0% vs. 8.4%). Deaths resulting from adverse events, none of which were deemed by the investigators to be related to the trial regimen, were reported less frequently in the Compound A group than in the placebo group (Table 11). After day 29, three additional deaths resulting from adverse events occurred in the placebo group, as compared with one additional death reported in the Compound A group.

TABLE 11
Incidences of Adverse Events in the Safety Population

| Adverse Events | | | |
|---|---|---|---|
| and | Compound A | Placebo | Estimated Difference |
| Discontinuation | (N = 710) | (N = 701) | (95% CI)* |
| | number (percent) | | percentage points |
| Participants with | | | |
| adverse events | | | |
| ≥1 Adverse event | 216 (30.4) | 231 (33.0) | −2.5 (−7.4 to 2.3) |
| ≥1 Adverse event | 57 (8.0) | 59 (8.4) | −0.4 (−3.3 to 2.5) |
| related to the | | | |
| assigned | | | |
| regiment† | | | |
| ≥1 Serious | 49 (6.9) | 67 (9.6) | −2.7 (−5.6 to 0.2) |
| adverse event | | | |
| ≥1 Serious | 0 | 1 (0.1) | −0.1 (−0.8 to 0.4) |
| adverse event | | | |
| related to the | | | |
| assigned | | | |
| regiment† | | | |
| Death | 2 (0.3) | 12 (1.7) | −1.4 (−2.7 to −0.5) |
| Participants who | | | |
| discontinued | | | |
| the assigned | | | |
| regimen | | | |
| because of an | | | |
| adverse event | | | |
| Adverse event | 10 (1.4) | 20 (2.9) | −1.4 (−3.1 to 0.1) |
| Adverse event | 4 (0.6) | 3 (0.4) | 0.1 (−0.8 to 1.1) |
| related to the | | | |
| assigned | | | |
| regiment† | | | |
| Serious adverse | 5 (0.7) | 13 (1.9) | −1.2 (−2.5 to 0.0) |
| event | | | |
| Serious adverse | 0 | 0 | 0.0 (−0.5 to 0.5) |
| event related to | | | |
| the assigned | | | |
| regiment† | | | |

*Differences shown are for Compound A as compared with placebo. Difference estimates were based on the Miettinen and Nurminen method.
†Related events were those determined by the investigators to be related to the assigned regimen.

The most frequently reported adverse events (those that occurred in ≥2% of participants in either group) were Covid-19 pneumonia (which occurred in 6.3% of participants in the Compound A group as compared with 9.6% of those in the placebo group), diarrhea (2.3% vs. 3.0%), and bacterial pneumonia (2.0% vs. 1.6%); worsening of Covid-19 was reported as an adverse event in 7.9% as compared with 9.8%. The most frequently reported adverse events (occurring in ≥1% of participants in either group) that were deemed to be related to the trial regimen were diarrhea (1.7% vs. 2.1%), nausea (1.4% vs. 0.7%), and dizziness (1.0% vs. 0.7%). One participant each in the Compound A and placebo groups met the prespecified criteria for a postbaseline platelet count below 50,000 per microliter; the low platelet count in the Compound A-treated participant was reported on day 12 and was not deemed to be related to treatment.

### SUMMARY

A phase 3, double-blind, randomized, placebo-controlled trial evidenced the efficacy and safety of treatment with 800 mg of Compound A twice daily for 5 days, started within 5 days after the onset of signs or symptoms in nonhospitalized, unvaccinated adults with mild-to-moderate, laboratory-confirmed Covid-19 and at least one risk factor for severe Covid-19 illness. The primary efficacy end point was the incidence hospitalization or death at day 29; the incidence of adverse events was the primary safety end point.

A total of 1433 participants underwent randomization; 716 were assigned to receive Compound A and 717 to receive placebo. With the exception of an imbalance in sex, baseline characteristics were similar in the two groups.

The superiority of Compound A was demonstrated. The percentage of participants who were hospitalized or died through day 29 was lower in the Compound A group than in the placebo group (6.8% [48 of 709] vs. 9.7% [68 of 699]; difference, −3.0 percentage points; 95% confidence interval, −5.9 to −0.1), providing evidence of a substantial mortality benefit (89% decreased risk for death), a shorter time to resolution for most COVID-19 signs and symptoms, a greater reduction in mean viral load from baseline, and a lack of safety concerns compared with placebo through day 29.

Results of subgroup analyses were largely consistent with these overall results; in some subgroups, such as patients with evidence of previous SARS-CoV-2 infection, those with low baseline viral load, and those with diabetes, the point estimate for the difference favored placebo. One death was reported in the Compound A group and 9 were reported in the placebo group through day 29. Adverse events were reported in 216 of 710 participants (30.4%) in the Compound A group and 231 of 701 (33.0%) in the placebo group.

In conclusion, early treatment with Compound A reduced the risk of hospitalization or death in at-risk, unvaccinated adults with Covid-19.

### Example 4: Results from Additional Analyses of Data from Phase 3 Component of Trial in Example 3

Additional analyses were conducted to evaluate additional potential benefits of Compound A for the treatment of mild to moderate COVID-19 based on clinical markers and the need for respiratory interventions and medical services from the phase 3 component of the trial discussed in Example 3. Changes in high-sensitivity C-reactive protein (CRP) concentration, SpO₂, the need for respiratory interventions, acute care visits, and COVID-19-related acute care visits were evaluated in all randomly assigned participants who received Compound A or placebo as described in Example 3. Respiratory interventions plus time to discharge were assessed in the subgroup of participants who were hospitalized after randomization.

Baseline demographic and clinical characteristics and mean changes in CRP concentration and SpO₂ from baseline through day 29 were evaluated in the safety population (Table 12), which consisted of all participants who had undergone randomization and had received at least 1 dose of study drug. The use of any respiratory interventions (including conventional oxygen therapy, a high-flow heated and humidified device, noninvasive mechanical ventilation, and invasive mechanical ventilation), acute care visits, and COVID-19-related acute care visits were assessed in the modified intention-to-treat (MITT) population. The MITT population included all participants who were randomly assigned, received at least 1 dose of study drug, and were not hospitalized before the first dose of study drug. Participants hospitalized before the first dose were not included in the MITT population because they could not be assessed for the primary efficacy end point. Respiratory interventions along with time to hospital discharge were examined in participants in the MITT population who required hospitalization after randomization.

TABLE 12
Baseline Demographic and Clinical Characteristics
in Safety Population

| | Compound A | Placebo |
|---|---|---|
| | (n = 710) | (n = 701) |
| Female, n (%) | 380 (53.5) | 345 (49.2) |
| Median age (range) | 43 (18-90) | 44 (18-88) |
| Median time from symptom onset to | 4 (1-5) | 4 (1-5) |
| randomization (range) | | |
| Race, n (%) | | |
| White | 397 (55.9) | 405 (57.8) |
| Multiple | 189 (26.6) | 197 (28.1) |
| American Indian or Alaska Native | 60 (8.5) | 43 (6.1) |
| Black or African American | 39 (5.5) | 34 (4.9) |
| Asian | 25 (3.5) | 22 (3.1) |
| Hispanic or Latino, n (%) | 352 (49.6)* | 348 (49.6)^{†} |
| Region, n (%) | | |
| Latin America | 330 (46.5) | 323 (46.1) |
| Europe | 229 (32.3) | 233 (33.2) |
| Africa | 90 (12.7) | 84 (12.0) |
| North America | 42 (5.9) | 45 (6.4) |
| Asia Pacific | 19 (2.7) | 16 (2.3) |
| Risk factors for severe COVID-19, n (%) | | |
| At least one risk factor | 709 (99.9) | 698 (99.6) |
| Body mass index ≥30 | 535 (75.4) | 508 (72.5) |
| Age >60 years | 119 (16.8) | 127 (18.1) |
| Diabetes mellitus | 107 (15.1) | 118 (16.8) |
| Serious heart condition | 86 (12.1) | 78 (11.1) |
| Chronic kidney disease | 38 (5.4) | 44 (6.3) |
| Chronic obstructive pulmonary disease | 22 (3.1) | 34 (4.9) |
| Active cancer | 13 (1.8) | 16 (2.3) |
| COVID-19 severity, n (%) | | |
| Mild | 395 (55.6) | 378 (53.9) |
| Moderate | 312 (43.9) | 321 (45.8) |
| Severe | 3 (0.4) | 1 (0.1) |
| Unknown^{‡} | 0 (0.0) | 1 (0.1) |
| SARS-CoV-2 RNA qualitative assay | | |
| Detectable | 615 (86.6) | 615 (87.7) |
| Undetectable | 54 (7.6) | 51 (7.3) |
| Unknown^{‡} | 41 (5.8) | 35 (5.0) |
| SARS-CoV-2 RNA quantitative assay, | | |
| n (%) | | |
| High (>1,000,000 copies/mL) | 389 (54.8) | 383 (54.6) |
| Low (500 to ≤1,000,000 copies/mL) | 161 (22.7) | 163 (23.3) |
| Undetectable (<500 copies/mL) | 64 (9.0) | 71 (10.1) |
| Unknown^{‡} | 96 (13.5) | 84 (12.0) |
| SARS-CoV-2 nucleocapsid antibody, | | |
| n (%) | | |
| Positive | 137 (19.3) | 147 (21.0) |
| Negative | 541 (76.2) | 521 (74.3) |
| Unknown^{‡} | 32 (4.5) | 33 (4.7) |

*Data were not reported or were missing in 6 participants.
^{†}Data were not reported or were missing in 3 participants.
^{‡}Includes missing data, invalid samples, tests not done, or results reported as unknown.

### Results

Changes from Baseline in High-Sensitivity C-Reactive Protein (CRP) Concentration

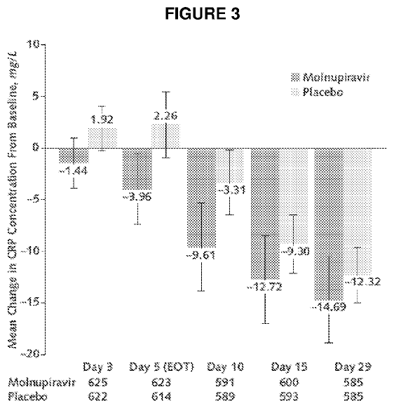

In the safety population, participants who received Compound A had earlier and larger reductions in mean change from baseline in CRP values at all postbaseline visits than those who received placebo. In the Compound A group, a reduction in mean CRP values was evident as early as day 3 (the first postbaseline visit) and continued through day 29, whereas in the placebo group, a reduction was not seen until day 10 (FIG. 3).

Changes from Baseline in Oxygen Saturation (SpO₂)

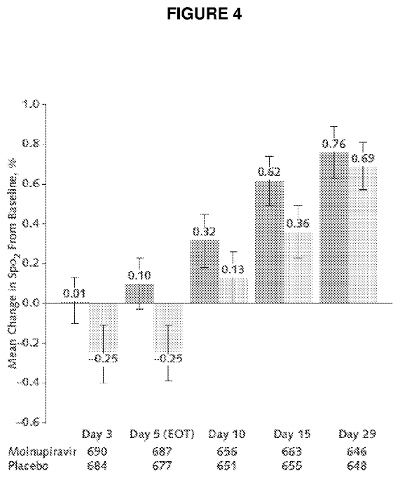

Compound A-treated participants had earlier and larger improvements in mean change from baseline in SpO₂ values compared with placebo recipients at all postbaseline visits in the safety population. Improvement in mean SpO₂ was observed as early as day 3 and continued through day 29 in the Compound A group, whereas placebo recipients did not show an increase in mean SpO₂ until day 10. (FIG. 4).

### Relative Risk Reduction (RRR)

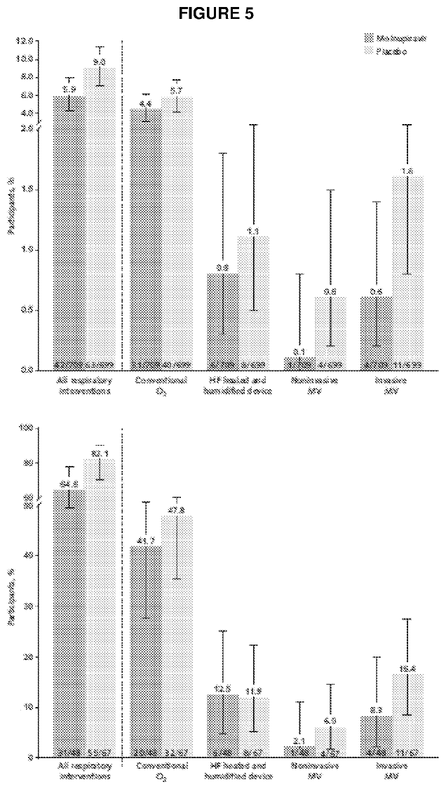

In the MITT population, fewer Compound A-treated participants required use of respiratory interventions (FIG. 5, top), with an RRR of 34.3% (CI, 4.3% to 54.9%) for all respiratory interventions compared with placebo recipients. The RRRs were 23.6% (CI, −20.7% to 51.6%) for oxygen therapy with conventional oxygen; 26.1% (CI, −112% to 74.2%) for a high-flow heated and humidified device; 75.4% (CI, −120% to 97.2%) for noninvasive mechanical ventilation; and 64.1% (CI, −12.1% to 88.5%) for invasive mechanical ventilation.

A reduction in the use of respiratory interventions was also observed in the subgroup of participants who required hospitalization after randomization (FIG. 5, bottom). Compared with placebo recipients, this subgroup had RRRs of 21.3% (CI, 0.2% to 38.0%) for all respiratory interventions, 12.8% (CI, −32.5% to 42.6%) for conventional oxygen, −4.7% (CI, −182.2% to 61.2%) for a high-flow heated and humidified device; 65.1% (CI, −202.5% to 96.0%) for noninvasive mechanical ventilation; and 49.2% (CI, −49.9% to 82.8%) for invasive mechanical ventilation. In this subgroup of hospitalized participants, the median time to hospital discharge was 9 days (CI, 7 to 12 days) in the Compound A group and 12 days (CI, 9 to 14 days) in the placebo group.

### Acute Care Visits

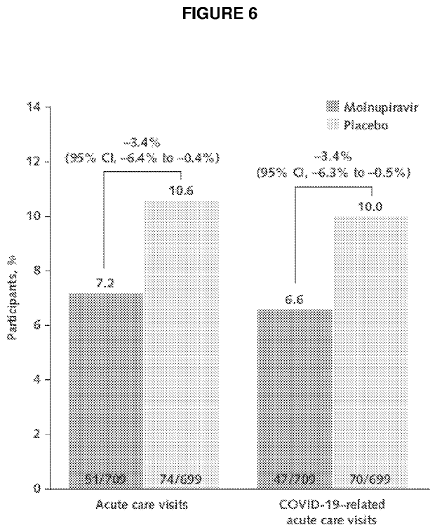

In the MITT population, prespecified analyses showed that the proportion of participants who had an acute care visit or a COVID-19-related acute care visit was lower in the Compound A group (7.2% and 6.6%, respectively) than in the placebo group (10.6% and 10.0%, respectively) (FIG. 6), with RRRs of 32.1% (CI, 4.4% to 51.7%) and 33.8% (CI, 5.6% to 53.6%), respectively.

### Example 5: PK of Compound A

A population pharmacokinetic (popPK) analysis for Compound A exposure was conducted using 4202 NHC plasma concentrations collected in 1207 individuals from a phase 1 study in healthy participants, a phase 2a study in non-hospitalized participants with COVID-19, a Phase 2 study in hospitalized participants with COVID-19, and a Phase 2/3 study in non-hospitalized participants with COVID-19. All studies were randomized, double-blind, placebo-controlled studies of safety, tolerability, and PK of Compound A after oral administration.

### Results

Compound A PK was best described by a 2-compartment model with a transit-compartment absorption model and linear elimination. Compound A apparent elimination clearance increased with body weight less-than-proportionally (power 0.412) and was estimated as 70.6 L/h in 80-kg individuals with a moderate inter-individual variability (43.4% coefficient of variation). Additionally, effects of sex and body mass index on apparent central volume and food status and formulation on the absorption mean transit time were identified as statistically significant descriptor of variability in these PK parameters. However, none of the identified covariate effects caused clinically relevant changes in the area under the NHC concentration versus time curve between doses, the exposure metric most closely related to clinical response. Overall, the popPK model indicates that Compound A can be administered in adults without dose adjustment based on age, sex, body size, food, and mild-to-moderate renal or mild hepatic impairment.

It will be appreciated that various of the above-discussed and other features and functions, or alternatives thereof, may be desirably combined into many other different systems or applications. It will also be appreciated that various presently unforeseen or unanticipated alternatives, modifications, variations, or improvements therein may be subsequently made by those skilled in the art are also intended to be encompassed by the following claims.

1. A method of treating SARS-CoV-2 in a subject in need thereof, comprising administering {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate or a prodrug thereof to the subject twice daily for 1 to 10 days.

2. (canceled)

3. The method of claim 1, wherein treating SARS-CoV-2 in a subject in need thereof comprises reducing the risk of death due to SARS-CoV-2 in a subject in need thereof, wherein the risk of death due to SARS-CoV-2 is reduced compared to risk of death due to SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

4. The method of claim 1, wherein treating SARS-CoV-2 in a subject in need thereof comprises reducing the risk of hospitalization due to SARS-CoV-2 in a subject in need thereof, wherein the risk of hospitalization due to SARS-CoV-2 is reduced compared to risk of hospitalization due to SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

5. The method of claim 1, wherein treating SARS-CoV-2 in a subject in need thereof comprises reducing time to SARS-CoV-2 RNA clearance in a subject in need thereof, wherein the time to SARS-CoV-2 RNA clearance is reduced compared to time to SARS-CoV-2 RNA clearance in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

6. The method of claim 1, wherein treating SARS-CoV-2 in a subject in need thereof comprises reducing SARS-CoV-2 viral load in a subject in need thereof, wherein the viral load of SARS-CoV-2 is reduced compared to SARS-CoV-2 viral load in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

7. The method of claim 1, wherein treating SARS-CoV-2 in a subject in need thereof comprises reducing morbidity or mortality due to SARS-CoV-2 in a subject in need thereof, wherein the morbidity or mortality due to SARS-CoV-2 is reduced compared to morbidity or mortality due to SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

8. The method of claim 1, wherein treating SARS-CoV-2 in a subject in need thereof comprises reducing time to normalization of high-sensitivity C-reactive protein in a subject with SARS-CoV-2, wherein the time to normalization of high-sensitivity C-reactive protein in a subject with SARSCoV-2 is reduced compared to time to normalization of high-sensitivity C-reactive protein in a subject with SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

9. The method of claim 1, wherein treating SARS-CoV-2 in a subject in need thereof comprises reducing time to normalization of oxygen saturation in a subject with SARS-CoV-2, wherein the time to normalization of oxygen saturation due to SARS-CoV-2 is reduced compared to time to normalization of oxygen saturation in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

10. The method of claim 1, wherein treating SARS-CoV-2 in a subject in need thereof comprises reducing need for respiratory interventions in a subject with SARS-CoV-2, wherein the need for respiratory interventions in a subject with SARS-CoV-2 is reduced compared to need for respiratory interventions in a subject with SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

11. The method of claim 1, wherein treating SARS-CoV-2 in a subject in need thereof comprises reducing length of hospitalization in a subject with SARS-CoV-2, wherein the length of hospitalization in a subject with SARS-CoV-2 is reduced compared to length of hospitalization in a subject with SARS-CoV-2 in the absence of administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate.

12. The method of claim 1, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is administered to the subject orally.

13. The method of claim 1, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is administered to the subject twice daily, as individual doses in an amount of from about 200 mg to about 1200 mg.

14. The method of claim 13, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is administered twice daily, as individual doses of about 800 mg.

15. The method of claim 1, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is administered twice daily for 5 days.

16. The method of claim 1, wherein administration of {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is initiated within 5 days of symptom onset.

17. The method of claim 1, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is provided as individual doses using one, two, three, or four 200 mg capsules.

18. The method of claim 14, wherein {(2R,3S,4R,5R)-3,4-dihydroxy-5-[(4Z)-4-(hydroxyimino)-2-oxo-3,4-dihydropyrimidin-1 (2H)-yl]oxolan-2-yl}methyl 2-methylpropanoate is provided as individual doses using four 200 mg capsules.

19. The method of claim 1, wherein said method results in a reduction in risk of hospitalization or death for the subject.

20. The method of claim 19, wherein said method results in a reduction in risk of hospitalization or death for the subject of 1 to 10 percent.

21. The method of claim 19, wherein said method results in a relative reduction in risk of hospitalization or death for the subject of up to about 50 percent.

22. The method of claim 1, wherein said subject is determined to be at increased risk for severe illness from COVID-19.

23. The method of claim 22, wherein said subject has one or more underlying medical condition associated with being at increased risk for severe illness from COVID-19.

24. The method of claim 23, wherein the at least one risk factor is chosen from age, active cancer, chronic kidney disease, chronic obstructive pulmonary disease, obesity, serious heart conditions, and diabetes mellitus.

25. The method of claim 1, wherein the subject does not have at least one exclusion criterion chosen from anticipated need for hospitalization for COVID-19 within the next 48 hours, dialysis or estimated glomerular filtration rate less than 30 ml per minute per 1.73 m², pregnancy, unwillingness to use contraception during the intervention period and for 5 at least 4 days after completion of the regimen, severe neutropenia, platelet count below 100,000 per microliter, and SARS-CoV-2 vaccination.
